(19) United States
(12) Patent Application Publication
(10) Pub. No.: US 2011/0232530 A1
(43) Pub. Date: Sep. 29, 2011

(54) FLUORINATED ETHER URETHANES AND METHODS OF USING THE SAME

(76) Inventors: Rudolf J. Dams, Antwerp (BE); Miguel A. Guerra, Woodbury, MN (US); Klaus Hintzer, Kastl (DE); Michael Jurgens, Neuoetting (DE); Harald Kaspar, Burgkircheng (DE); Kai Helmut Lochhaas, Neuoetting (DE); Andreas R. Maurer, Langenneufnach (DE); Zai-Ming Qiu, Woodbury, MN (US); Werner Schwertfeger, Langgons (DE); Tilman C. Zipplies, Burghausen (DE)

(21) Appl. No.: 13/130,856

(22) PCT Filed: Nov. 23, 2009
(86) PCT No.: PCT/US09/65454

§ 371 Date: May 24, 2011

(30) Foreign Application Priority Data
Nov. 25, 2008 (US) 61/117779

(51) Int. Cl. C09D 7/12; C07C 237/06; C07F 7/10

(52) U.S. Cl. 106/287.11; 564/198; 556/419; 106/287.25

(57) ABSTRACT

Compounds comprising end groups represented by formula (Rf-Q)ₐ-X-(A-)_{b} and compounds comprising the reaction product of components comprising a multifunctional isocyanate compound and a fluorinated compound represented by formula (Rf-Q)ₐ-X—(Z)_{b}. Each Rf is independently a partially fluorinated or fully fluorinated group selected from Rf^{A}(O)₂CHL′-(CF₂)ₙ—; [Rf^{B}—(O)₁—C(L)H—CF₂—O]ₘ—W—; CF₃CFH—O—(CF₂)ₚ—; CF₃—(O—CF₂)Z—; and CF₃—O—(CF₂)₃—O—CF—. Methods of making an article having a surface using the fluorinated compounds and treated articles are also disclosed.

### CROSS REFERENCE TO RELATED APPLICATION

This application claims the benefit of U.S. Provisional Patent Application No. 61/117,779, filed Nov. 25, 2008, the disclosure of which is incorporated by reference herein in its entirety.

### BACKGROUND

Fluorochemicals have been used in a variety of applications for many years. For example, fluorochemicals have been used to provide properties such as hydrophobicity, oleophobicity, and stain resistance to various materials (e.g., ceramics, metals, fabrics, plastics, and porous stones). The particular properties provided depend, for example, on the particular composition of the fluorochemical and the particular material treated with the fluorochemical.

Traditionally, many widely used fluorinated repellents include long-chain perfluoroalkyl groups, (e.g., perfluorooctyl groups). Recently, however, there has been an industry trend away from using perfluorooctyl fluorochemicals, which has resulted in a desire for new types of surface treatments that provide hydrophobicity, olephobicity, and stain resistance and may be used in a variety of applications.

### SUMMARY

The present disclosure provides compounds that have partially fluorinated polyether groups and/or fully have fluorinated polyether groups with a low number (e.g., up to 4) continuous perfluorinated carbon atoms. The compounds may be useful, for example, as water- and oil-repellent surface treatments. Manufacturing fluorinated materials is typically expensive, and the cost increases with the number of fluorine atoms. Applicants have found compounds that have high fluorine efficiency (i.e., the compounds provide properties that would be expected from compounds having a higher number of fluorine atoms). In some embodiments, for example, the compounds disclosed herein unexpectedly raise the contact angle versus water and/or hexadecane to an extent comparable to treatment compounds having a greater number of perfluorinated carbon atoms. In other embodiments, the compounds disclosed herein unexpectedly raise the contact angle versus water and/or hexadecane to an extent higher than treatment compounds having the same number of perfluorinated carbon atoms, but in a different configuration. The fluorine efficiency of the compounds disclosed herein may provide advantages in manufacturing cost.

In one aspect, the present disclosure provides a compound comprising:
an end group represented by formula:

(Rf-Q)ₐ-X-(A-)_{b}; and
a multivalent unit comprising a segment represented by formula:

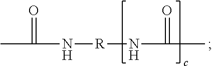

wherein
each Rf is independently:

Rf^{A}—(O)ᵣ—CHL′-(CF₂)ₙ—;

[Rf^{B}—(O)ₜ—C(L)H—CF₂—O]ₘ—W—;

CF₃CFH—O—(CF₂)ₚ—;

CF₃—(O—CF₂)_{z}—; or

CF₃—O—(CF₂)₃—O—CF₂—;
each Q is independently alkylene or arylalkylene, wherein alkylene and arylalkylene are optionally interrupted or terminated by at least one functional group that is independently ether, amine, ester, amide, carbamate, or urea, wherein when “a” is 1, Q may also be a bond, —C(O)O—, or —C(O)—N(R″)—;
X is alkylene or an alkylenic polymer backbone, each of which is optionally interrupted by —S— or —O—, wherein the alkylenic polymer backbone is optionally substituted with at least one alkyl ester group that is optionally substituted with —Si(G)₃, an ammonium group, a polyalkyleneoxy segment, a carboxylate, a sulfonate, a sulfate, a phosphate, or a phosphonate, wherein each G is independently hydroxyl, alkoxy, acyloxy, aryloxy, halogen, alkyl, or phenyl, and wherein at least one G is alkoxy, acyloxy, aryloxy, or halogen;
each A is independently —O—, —N(R¹)—, —S—, or —C(O)O—;
R″ and R¹ are each independently hydrogen or alkyl having up to 4 carbon atoms;
a is a number from 1 to 10;
b is a number from 1 to 5;
c is a number from 1 to 20;
Rf^{A} and Rf^{B} independently represent a partially or fully fluorinated alkyl group having from 1 to 6 carbon atoms and optionally interrupted with at least one oxygen atom;
L is F or CF₃;
L′ is F or H;
W is alkylene or arylene;
r is 0 or 1, wherein when r is 0, then Rf^{A} is interrupted with at least one oxygen atom;
t is 0 or 1;
m is 1, 2, or 3;
n is 0 or 1;
each p is independently a number from 1 to 6;
z is a number from 2 to 7; and
R is alkylene, arylene, or arylalkylene, each of which is optionally interrupted by at least one biruet, allophanate, uretdione, or isocyanurate linkage.
   In some embodiments, the compound further comprises an end group represented by formula:

alkyl-A-;

Rf³-Q¹-A-;

alkyl-O-[EO]_{f}—[R²O]_{g}-[EO]_{f}—;

alkyl-O—[R²O]_{g}-[EO]_{f}—[R²O]_{g}—;

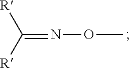

[(G)₃Si]_{d}—X′-A-;

(D)₁₋₃-R³-A-; or

(M)₁₋₂-R⁴-A-;

wherein
Rf³ is perfluoroalkyl having up to 6 carbon atoms and optionally interrupted by one or two —O— groups;
Q¹ is alkylene or arylalkylene, wherein alkylene and arylalkylene are optionally interrupted or terminated by at least one functional group that is independently ether, amine, ester, amide, sulfonamide, carbamate, or urea;
EO represents —CH₂CH₂O—;
each R²O independently represents —CH(CH₃)CH₂O—, —CH₂CH(CH₃)O—, —CH₂CH₂CH₂O—, —CH(CH₂CH₃)CH₂O—, —CH₂CH(CH₂CH₃)O—, —CH₂CH₂CH₂CH₂O—, or —CH₂C(CH₃)₂O—;
each f is independently a number from 1 to 150;
each g is independently a number from 0 to 55;
each G is independently hydroxyl, alkoxy, acyloxy, aryloxy, halogen, alkyl, or phenyl, wherein at least one G is alkoxy, acyloxy, aryloxy, or halogen;
each D is independently acrylate, methacrylate, epoxide, glycidoxy, or vinyl;
M is an ammonium group, a carboxylate, a sulfonate, a sulfate, a phosphate, or a phosphonate;
each R′ is independently hydrogen, alkyl, or aryl;
R³ is divalent, trivalent, or tetravalent alkylene;
R⁴ is alkylene that is optionally interrupted by at least one ether linkage or amine linkage;
X′ is alkylene or an alkylenic polymer backbone, each of which is optionally interrupted by —S— or —O—, wherein the alkylenic polymer backbone is optionally substituted with at least one alkyl ester group that is optionally substituted with an ammonium group, a carboxylate, a sulfonate, a sulfate, a phosphate, or a phosphonate; and
d is a number from 1 to 5.
   In some embodiments, the compound is represented by formula:

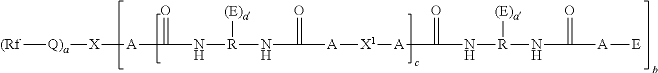

wherein

each a′ is independently 0, 1, or 2;

e is a number from 0 to 20;

X¹ is alkylene, polyalkyleneoxy, fluoroalkylene, or polyfluoroalkyleneoxy, wherein alkylene is optionally interrupted by at least one of —O—, polydialkylsiloxane, polydiarylsiloxane, or polyalkylarylsiloxane and is optionally substituted with —Si(G)₃, an ammonium group, a polyalkyleneoxy segment, a carboxylate, a sulfonate, a sulfate, a phosphate, or a phosphonate;

each E is independently an end group represented by formula:

(Rf-Q)ₐ-X-A-;

alkyl-A-;

Rf³-Q¹-A-;

alkyl-O[EO]_{f}—[R²O]_{g}-[EO]_{f}—;

alkyl-O—[R²O]_{g}-[EO]_{f}—[R²O]_{g}—;

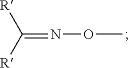

[(G)₃Si]_{d}—X′-A-;

(D)₁₋₃-R³-A-; or

(M)₁₋₂-R⁴-A-; wherein
Rf³ is perfluoroalkyl having up to 6 carbon atoms and optionally interrupted by one or two —O— groups;
Q¹ is alkylene or arylalkylene, wherein alkylene and arylalkylene are optionally interrupted or terminated by at least one functional group that is independently ether, amine, ester, amide, sulfonamide, carbamate, or urea;
EO represents —CH₂CH₂O—;
each R²O independently represents —CH(CH₃)CH₂O—, —CH₂CH(CH₃)O—, —CH₂CH₂CH₂O—, —CH(CH₂CH₃)CH₂O—, —CH₂CH(CH₂CH₃)O—, —CH₂CH₂CH₂CH₂O—, or —CH₂C(CH₃)₂O—;
each f is independently a number from 1 to 150;
each g is independently a number from 0 to 55;
each G is independently hydroxyl, alkoxy, acyloxy, aryloxy, halogen, alkyl, or phenyl, wherein at least one G is alkoxy, acyloxy, aryloxy, or halogen;
each D is independently acrylate, methacrylate, epoxide, glycidoxy, or vinyl;
M is an ammonium group, a carboxylate, a sulfonate, a sulfate, a phosphate, or a phosphonate;
each R′ is independently hydrogen, alkyl, or aryl;
R³ is divalent, trivalent, or tetravalent alkylene;
R⁴ is alkylene that is optionally interrupted by at least one ether linkage or amine linkage;
X′ is alkylene or an alkylenic polymer backbone, each of which is optionally interrupted by —S— or —O—, wherein the alkylenic polymer backbone is optionally substituted with at least one alkyl ester group that is optionally substituted with an ammonium group, a carboxylate, a sulfonate, a sulfate, a phosphate, or a phosphonate; and
d is a number from 1 to 5.

In another aspect, the present disclosure provides a compound comprising a reaction product of components comprising a multifunctional isocyanate compound and a fluorinated compound represented by formula:

(Rf-Q)ₐ-X—(Z)_{b}

wherein
each Rf is independently:

Rf^{A}—(O)ᵣ—CHL′-(CF₂)ₙ—;

[Rf^{B}—(O)ₜ—C(L)H—CF₂—O]ₘ—W—;

CF₃CFH—O—(CF₂)ₚ—;

CF₃—(O—CF₂)_{z}—; or

CF₃O—(CF₂)₃—O—CF₂—;
each Q is independently alkylene or arylalkylene, wherein alkylene and arylalkylene are optionally interrupted or terminated by at least one functional group that is independently ether, amine, ester, amide, carbamate, or urea, wherein when “a” is 1, Q may also be a bond, —C(O)O—, or —C(O)—N(R″)—, and wherein R″ is hydrogen or alkyl having up to 4 carbon atoms;
X is alkylene or an alkylenic polymer backbone, each of which is optionally interrupted by —S— or —O—, wherein the alkylenic polymer backbone is optionally substituted with at least one alkyl ester group that is optionally substituted with —Si(G)₃, an ammonium group, a polyalkyleneoxy segment, a carboxylate, a sulfonate, a sulfate, a phosphate, or a phosphonate, wherein each G is independently hydroxyl, alkoxy, acyloxy, aryloxy, halogen, alkyl, or phenyl, and wherein at least one G is alkoxy, acyloxy, aryloxy, or halogen;
each Z is independently hydroxyl, amino, mercaptan, isocyanate, epoxy, or a carboxylic acid;
a is a number from 1 to 10;
b is a number from 1 to 5;
Rf^{A} and Rf^{B} independently represent a partially or fully fluorinated alkyl group having from 1 to 6 carbon atoms and optionally interrupted with at least one oxygen atom;
L is F or CF₃;
L′ is F or H;
W is alkylene or arylene;
r is 0 or 1, wherein when r is 0, then Rf^{A} is interrupted with at least one oxygen atom;
t is 0 or 1;
m is 1, 2, or 3;
n is 0 or 1;
each p is independently a number from 1 to 6; and
z is a number from 2 to 7.

In another aspect, the present disclosure provides a method of making an article having a surface, the method comprising treating the surface with a compound disclosed herein. In some embodiments, the surface comprises at least one of fabric, textiles, carpets, leather, paper, ceramic (i.e., glasses, crystalline ceramics, glass ceramics, and combinations thereof), natural stone (e.g., sandstone, limestone, marble, and granite), concrete, masonry, man-made stone (i.e., engineered stone such as concrete), grout, metals, plastics, or wood. In some embodiments, the surface is a siliceous surface.

In another aspect, the present disclosure provides an article comprising a surface, wherein at least a portion of the surface is treated with a compound disclosed herein. In some embodiments, the surface comprises at least one of fabric, textiles, carpets, leather, paper, ceramic (i.e., glasses, crystalline ceramics, glass ceramics, and combinations thereof), natural stone (e.g., sandstone, limestone, marble, and granite), concrete, masonry, man-made stone (i.e., engineered stone such as concrete), grout, metals, plastics, or wood. In some embodiments, the surface is a siliceous surface.

In this application:

The terms “a”, “an”, and “the” are used interchangeably with the term “at least one”.

The term “comprises at least one of” or “at least one of” followed by a list of items refers to comprising any one of the items in the list and any combination of two or more items.

“Alkyl group” and the prefix “alk-” are inclusive of both straight chain and branched chain groups and of cyclic groups. In some embodiments, alkyl groups have up to 30 carbons (in some embodiments, up to 20, 15, 12, 10, 8, 7, 6, or 5 carbons) unless otherwise specified. Cyclic groups can be monocyclic or polycyclic and, in some embodiments, have from 3 to 10 ring carbon atoms.

“Alkylene” refers to a multivalent (e.g., divalent, trivalent, or tetravalent) form of the “alkyl” groups defined above.

“Arylalkylene” refers to an “alkylene” moiety to which an aryl group is attached.

The term “aryl” as used herein includes carbocyclic aromatic rings or ring systems, for example, having 1, 2, or 3 rings and optionally containing at least one heteroatom (e.g., O, S, or N) in the ring. Examples of aryl groups include phenyl, naphthyl, biphenyl, fluorenyl as well as furyl, thienyl, pyridyl, quinolinyl, isoquinolinyl, indolyl, isoindolyl, triazolyl, pyrrolyl, tetrazolyl, imidazolyl, pyrazolyl, oxazolyl, and thiazolyl.

The phrase “interrupted by at least one functional group”, for example, with regard to an alkyl (which may or may not be fluorinated), alkylene, or arylalkylene refers to having part of the alkyl, alkylene, or arylalkylene on both sides of the functional group.

The term “multivalent” means connecting at least two groups.

The term “polymeric” refers a molecule having a structure that includes the multiple repetition of units derived, actually or conceptually, from molecules of low relative molecule mass. The term “polymeric” includes “oligomeric”.

The term “urethane” refers to a compound having more than one carbamate, urea, biuret, allophanate, uretdione, or isocyanurate linkage in any combination.

In this application, all numerical ranges are inclusive of their endpoints and nonintegral values between their endpoints unless otherwise stated.

### DETAILED DESCRIPTION

For end groups represented by formula (Rf-Q)ₐ-X-(A-)_{b} and fluorinated compounds represented by formula (Rf-Q)ₐ-X—(Z)_{b}, each Rf is independently:

Rf^{A}—(O)ᵣ—CHL′-(CF₂)ₙ— I;

[Rf^{B}—(O)ₜ—C(L)H—CF₂—O]ₘ—W— II;

CF₃CFH—O—(CF₂)ₚ— III;

CF₃—(O—CF₂)_{z}— IV; or

CF₃—O—(CF₂)₃—O—CF₂— V.

In some embodiments of end groups represented by formula (Rf-Q)ₐ-X-(A-)_{b} and fluorinated compounds represented by formula (Rf-Q)ₐ-X—(Z)_{b}, each Rf is partially fluorinated and is independently Rf^{A}—(O)ᵣ—CHF—(CF₂)ₙ—, [Rf^{B}—(O)ₜ—C(L)H—CF₂—O]ₘ—W—, or CF₃CFH—O—(CF₂)ₚ—. It has been found, surprisingly, that in some embodiments, compounds comprising these partially fluorinated segments are effective oil- and water-repellent surface treatments in comparison to compounds containing fully fluorinated segments. In other embodiments of end groups represented by formula (Rf-Q)ₐ-X-(A-)_{b} and fluorinated compounds represented by formula (Rf-Q)ₐ-X—(Z)_{b}, each Rf is independently CF₃CFH—O—(CF₂)ₚ—, CF₃—(O—CF₂)_{z}— or CF₃—O—(CF₂)₃—O—CF₂—. In other embodiments, each Rf is independently CF₃—(O—CF₂)_{z}— or CF₃—O—(CF₂)₃—O—CF₂—. In some embodiments, the compounds disclosed herein comprising these fully fluorinated Rf segments unexpectedly raise the contact angle versus water and/or hexadecane to an extent higher than treatment compounds having the same number of perfluorinated carbon atoms, but in a different configuration.

In some embodiments of end groups represented by formula (Rf-Q)ₐ-X-(A-)_{b} and fluorinated compounds represented by formula (Rf-Q)ₐ-X—(Z)_{b}, Rf has a molecular weight of up to 600 grams per mole (in some embodiments, up to 500, 400, or even up to 300 grams per mole).

In Formulas I and II, Rf^{A} and Rf^{B} independently represent a partially or fully florinated alkyl group having from 1 to 6 (in some embodiments, 5, 4, 3, 2, or 1) carbon atoms and optionally interrupted with at least one oxygen atom. Rf^{A} and Rf^{B} include linear and branched alkyl groups. In some embodiments, Rf^{A} and/or Rf^{B} is linear. In some embodiments, Rf^{A} and Rf^{B} independently represent fully fluorinated alkyl groups having from 1 to 3 carbon atoms. In some embodiments, Rf^{A} and Rf^{B} independently represent a fully fluorinated alkyl group interrupted with at least one oxygen atom, in which the alkyl groups between oxygen atoms have up to 3 (in some embodiments, 2 or 1) carbon atoms, and wherein the terminal alkyl group has up to 3 (in some embodiments, 2 or 1) carbon atoms. In some embodiments, Rf^{A} and Rf^{B} independently represent a partially fluorinated alkyl group having up to 6 (in some embodiments, 5, 4, 3, 2, or 1) carbon atoms and up to 2 hydrogen atoms. In some embodiments, Rf^{A} and Rf^{B} independently represent a partially fluorinated alkyl group having up to 2 hydrogen atoms and interrupted with at least one oxygen atom, in which the alkyl groups between oxygen atoms have up to 3 (in some embodiments, 2 or 1) carbon atoms, and wherein the terminal alkyl group has up to 3 (in some embodiments, 2 or 1) carbon atoms.

In some embodiments of Formulas I and II, Rf^{A} and Rf^{B} are independently represented by formula

R_{f}¹—[OR_{f}²]ₓ—.

R_{f}¹ is a perfluorinated alkyl group having from 1 to 3 (in some embodiments, 1 to 2) carbon atoms. Each R_{f}² is independently perfluorinated alkylene having from 1 to 3 carbon atoms. x is a value from 1 to 4. In some of these embodiments, t is 1, and r is 1.

In some embodiments of Formulas I and II, Rf^{A} and Rf^{B} are independently represented by formula

R_{f}⁴—[OR_{f}⁵]_{y}—O—CF₂—.

R_{f}⁴ is a perfluorinated alkyl group having from 1 to 3 (in some embodiments, 1 to 2) carbon atoms. Each R_{f}⁵ is independently perfluorinated alkylene having from 1 to 3 carbon atoms. y is a value from 0 to 4. In some of these embodiments, t is 0, and r is 0.

In some embodiments of Formulas I and II, Rf^{A} and Rf^{B} are independently represented by formula R_{f}⁷—(OCF₂)ₚ—, wherein p is from 1 to 6 (in some embodiments, 1 to 4 or 1 to 3), and R_{f}⁷ is selected from the group consisting of a partially fluorinated alkyl group having 1, 2, 3, 4, 5, or 6 carbon atoms and 1 or 2 hydrogen atoms and a fully fluorinated alkyl group having 1, 2, 3 or 4 carbon atoms.

In some embodiments of Formulas I and II, Rf^{A} and Rf^{B} are independently represented by formula R_{f}⁸—O—(CF₂)ₚ—, wherein p is from 1 to 6 (in some embodiments, 1 to 4 or 1 to 3) and R_{f}⁸ is selected from the group consisting of a partially fluorinated alkyl group having 1, 2, 3, 4, 5, or 6 carbon atoms and 1 or 2 hydrogen atoms and a fully fluorinated alkyl group having 1, 2, 3 or 4 carbon atoms.

In Formula II, L is selected from the group consisting of F and CF₃. In some embodiments of Formula II, L is F. In other embodiments, L is CF₃.

In Formula I, L′ is H or F. In some embodiments, L′ is F.

In Formula II, W is selected from the group consisting of alkylene and arylene. In some embodiments, alkylene includes linear, branched, and cyclic alkylene groups having from 1 to 10 (in some embodiments, 1 to 4) carbon atoms. In some embodiments, W is methylene. In some embodiments, W is ethylene. In some embodiments, arylene includes groups having 1 or 2 aromatic rings, optionally having at least one heteroatom (e.g., N, O, and S) in the ring, and optionally substituted with at least one alkyl group or halogen atom. In some embodiments, W is phenylene.

In Formula I, r is 0 or 1. In some embodiments, r is 1. In some embodiments, r is 0. In embodiments wherein r is 0, Rf^{A} is typically interrupted by at least one oxygen atom.

In Formula II, t is 0 or 1. In some embodiments, t is 1. In some embodiments, t is 0. In embodiments wherein t is 0, Rf^{B} is typically interrupted by at least one oxygen atom.

In Formula II, m is 1, 2, or 3. In some embodiments, m is 1.

In Formula I, n is 0 or 1. In some embodiments, n is 0. In some embodiments, n is 1.

In Formulas III, p is a number from 1 to 6 (i.e., 1, 2, 3, 4, 5, or 6). In some embodiments, p is 1, 2, 5, or 6. In some embodiments, p is 3. In some embodiments, p is 1 or 2. In some embodiments, p is 5 or 6.

In Formula IV, z is a number from 2 to 7 (i.e., 2, 3, 4, 5, 6, or 7). In some embodiments, z is a number from 2 to 6, 2 to 5, 2 to 4, 3 to 5, or 3 to 4.

In some embodiments, fluorinated compounds according to the present disclosure have an Rf group represented by Formula III (i.e., CF₃CFH—O—(CF₂)ₚ—). In some of these embodiments Rf is selected from the group consisting of CF₃CFH—O—(CF₂)₃— and CF₃CFH—O—(CF₂)₅—.

In some embodiments, fluorinated compounds according to the present disclosure have an Rf group represented by Formula I. In some of these embodiments, Rf is selected from the group consisting of:

C₃F₇—O—CHF—;

CF₃—O—CF₂CF₂—CF₂—O—CHF—;

CF₃CF₂CF₂—O—CF₂CF₂—CF₂—O—CHF—;

CF₃—O—CF₂—CF₂—O—CHF—;

CF₃—O—CF₂—O—CF₂—CF₂—O—CHF—;

CF₃—(O—CF₂)₂—O—CF₂—CF₂—O—CHF—; and

CF₃—(O—CF₂)₃—O—CF₂—CF₂—O—CHF—.

In other of these embodiments, Rf is selected from the group consisting of:

CF₃—O—CHF—CF₂—;

CF₃—O—CF₂—CF₂—O—CHF—CF₂—;

CF₃—CF₂—O—CHF—CF₂—;

CF₃—O—CF₂—CF₂—CF₂—O—CHF—CF₂—;

CF₃—O—CF₂—O—CF₂—CF₂—O—CHF—CF₂—;

CF₃—(O—CF₂)₂—O—CF₂—CF₂—O—CHF—CF₂—; and

CF₃—(O—CF₂)₃—O—CF₂—CF₂—O—CHF—CF₂—.

In other of these embodiments, Rf is selected from the group consisting of:

CF₃—O—CF₂—CHF—;

C₃F₇—O—CF₂—CHF—;

CF₃—O—CF₂—CF₂—CF₂—O—CF₂—CHF—;

CF₃—O—CF₂—O—CF₂—CF₂—O—CF₂—CHF—;

CF₃—(O—CF₂)₂—O—CF₂—CF₂—O—CF₂—CHF—; and

CF₃—(O—CF₂)₃—O—CF₂—CF₂—O—CF₂—CHF—.

In other of these embodiments, Rf is selected from the group consisting of:

CF₃—O—CF₂—CHF—CF₂—;

C₂F₅—O—CF₂—CHF—CF₂—;

C₃F₇—O—CF₂—CHF—CF₂—;

CF₃—O—CF₂—CF₂—CF₂—O—CF₂—CHF—CF₂—;

CF₃—O—CF₂—O—CF₂—CF₂—O—CF₂—CHF—CF₂—;

CF₃—(O—CF₂)₂—O—CF₂—CF₂—O—CF₂—CHF—CF₂—; and

CF₃—(O—CF₂)₃—O—CF₂—CF₂—O—CF₂—CHF—CF₂—.

In other of these embodiments, Rf is selected from the group consisting of:

CF₃—O—CF₂CF₂—CF₂—O—CHF—;

CF₃—O—CF₂—CF₂—CF₂—O—CHF—CF₂—;

CF₃—O—CF₂—CF₂—CF₂—O—CF₂—CHF—; and

CF₃—O—CF₂—CF₂—CF₂—O—CF₂—CHF—CF₂—.

In some embodiments, fluorinated compounds according to the present disclosure have an Rf group represented by Formula II. In some of these embodiments, L is F, m is 1, and W is alkylene (e.g., methylene or ethylene). In some of these embodiments, Rf is selected from the group consisting of:

CF₃—O—CHF—CF₂—O—CH₂—;

CF₃—O—CF₂—CF₂—CF₂—O—CHF—CF₂—O—CH₂;

C₃F₇—O—CHF—CF₂—O—CH₂—;

C₃F₇—O—CHF—CF₂—O—CH₂—CH₂—;

C₃F₇—O—CF₂—CF₂—O—CHF—CF₂—OCH₂—; and

C₃F₇—O—CF₂—CF₂—CF₂—O—CHF—CF₂—OCH₂—.

In other of these embodiments, Rf is represented by formula C₃F₇—O—CF₂—CHF—CF₂—OCH₂—. In other of these embodiments, Rf is selected from the group consisting of:

CF₃—CHF—CF₂—O—CH₂—; and

C₃F₇—CF₂—CHF—CF₂—OCH₂—.

In some embodiments, fluorinated compounds according to the present disclosure have an Rf group represented by Formula IV (i.e., CF₃—(O—CF₂)_{z}—). In some of these embodiments, z is a number from 2 to 6, 2 to 5, 2 to 4, 3 to 5, or 3 to 4.

In some embodiments, fluorinated compounds according to the present disclosure have an Rf represented by Formula V (i.e., CF₃—O—(CF₂)₃—O—CF₂—).

In fluorinated compounds represented by formula (Rf-Q)ₐ-X—(Z)_{b}, each Z is independently hydroxyl, amino, mercaptan, isocyanate, epoxy, or a carboxylic acid. In some embodiments, each Z is independently hydroxyl, amino, or isocyanate. In some embodiments, each Z is hydroxyl.

In end groups represented by formula (Rf-Q)ₐ-X-(A-)_{b}, each A is independently —O—, —N(R¹)—, —S—, or —C(O)O—, wherein R¹ is hydrogen or alkyl having up to 4 carbon atoms. In some embodiments, each A is independently —O— or —N(R¹)—. In some embodiments, R¹ is hydrogen. In some embodiments, each A is —O— (i.e., the end group is connected to the compound through a bond to oxygen).

In end groups represented by formula (Rf-Q)ₐ-X-(A-)_{b} and fluorinated compounds represented by formula (Rf-Q)ₐ-X—(Z)_{b}, each Q is independently alkylene or arylalkylene, wherein alkylene and arylalkylene are optionally interrupted or terminated by at least one functional group that is independently ether (i.e., —O—), amine (i.e., —N(R″)—), ester (i.e., —O—C(O)— or —C(O)—O—), amide (i.e., —N(R″)—C(O)— or —C(O)—N(R″)—), carbamate (i.e., —N(R″)—C(O)—O— or —O—C(O)—N(R″)—), or urea (i.e., —N(R″)—C(O)—N(R″)—), wherein when “a” is 1, Q may also be a bond, —C(O)O—, or —C(O)—N(R″)—, wherein R″ is hydrogen or alkyl having up to 4 carbon atoms. The phrase “interrupted by at least one functional group” refers to having alkylene or arylalkylene on either side of the functional group. The term “terminated by a functional group” refers to the functional group being connected to either the Rf group or the X group in formula (Rf-Q)ₐ-X-(A-)_{b} and (Rf-Q)ₐ-X—(Z)_{b}. In some embodiments, when “a” is 1, Q is selected from the group consisting of —C(O)—N(R″)- and —C(O)—O—. In some embodiments, Q is selected from the group consisting of a bond and —C(O)—N(R″)—. In some embodiments, Q is —C(O)—N(R″)—. In some embodiments, Q is a bond. In some embodiments, when “a” is greater than 1, Q is —C(O)—N(R″)-alkylene. In some embodiments, R″ is hydrogen or methyl. In some embodiments, R″ is hydrogen.

In end groups represented by formula (Rf-Q)ₐ-X-(A-)_{b} and fluorinated compounds represented by formula (Rf-Q)ₐ-X—(Z)_{b}, X is alkylene or an alkylenic polymer backbone, each of which is optionally interrupted by —S— or —O—, wherein the alkylenic polymer backbone is optionally substituted with at least one alkyl ester group that is optionally substituted with —Si(G)₃, an ammonium group, a polyalkyleneoxy segment, a carboxylate, a sulfonate, a sulfate, a phosphate, or a phosphonate, wherein each G is independently hydroxyl (i.e., —OH), alkoxy (e.g., —O-alkyl), acyloxy (e.g., —O—C(O)-alkyl), aryloxy (e.g., —O-aryl), halogen (i.e., fluoride, chloride, bromide, or iodine), alkyl (e.g., methyl or ethyl), or phenyl, and wherein at least one (e.g., two or three) G is alkoxy, acyloxy, aryloxy, or halogen. In some embodiments, alkoxy and acyloxy have up to 6 (or up to 4) carbon atoms, and the alkyl group is optionally substituted by halogen. In some embodiments, aryloxy has 6 to 12 (or 6 to 10) carbon atoms which may be unsubstituted or substituted by halogen, alkyl (e.g., having up to 4 carbon atoms), and haloalkyl. In some embodiments, each G is independently selected from the group consisting of is selected from the group consisting of halide (e.g., chloride) and alkoxy having up to ten carbon atoms. In some embodiments, each G is independently alkoxy having from 1 to 6 (e.g., 1 to 4) carbon atoms. In some embodiments, each G is independently methoxy or ethoxy. In some embodiments, “a” is 1, and X is alkylene having up to 10 (e.g., up to 8, 7, 6, 5, or 4) carbon atoms. In some embodiments, X is alkylene that is optionally interrupted by at least one ether group. In some embodiments, “a” is more than 1 (e.g., 10, 9, 8, 7, 6, 5, 4, 3, or 2), and X is an alkylenic polymer backbone. In some embodiments, b is more than 1 (e.g., 2, 3, or 4). In some embodiments, the alkylenic polymer backbone is substituted with at least one (e.g., at least 2, 3, or 5) alkyl ester group. In some embodiments, the alkylenic polymer backbone is represented by formula:

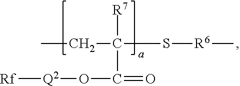

wherein
a is from 2 to 10 (e.g., 2, 3, 4, 5, 6, 7, 8, 9, or 10);
each Q² is independently —C(O)—N(R″)-alkylene, alkylene, —C(O)—N(R″)-arylalkylene, or arylalkylene, each of which is optionally interrupted by at least one ether linkage and wherein R″ is hydrogen or alkyl having up to 4 carbon atoms;
R⁶ is alkylene, arylene, or arylalkylene, each of which is divalent or trivalent and optionally interrupted by at least one ether linkage; and
each R⁷ is independently hydrogen or alkyl having up to 4 carbon atoms.
   In some embodiments, each R⁷ is independently hydrogen or methyl. Trivalent R⁶ groups may be substituted, for example, by two Z groups (e.g., hydroxyl groups).

In some embodiments, a and b are each 1, X is alkylene, and Q is a bond, —C(O)O—, or —C(O)—N(R″)—.

Fluorinated compounds represented by formula (Rf-Q)ₐ-X—(Z)_{b} in some embodiments are converted into end groups represented by formula (Rf-Q)ₐ-X-(A-)_{b} after a condensation reaction between the fluorinated compound and an isocyanate group. Fluorinated compounds represented by formula (Rf-Q)ₐ-X—(Z)_{b}, can be prepared, for example, starting with a partially or fully fluorinated carboxylic acid, a salt thereof, a carboxylic acid ester, or a carboxylic acid halide. Partially and fully fluorinated carboxylic acids and salts thereof, carboxylic acid esters, and carboxylic acid halides can be prepared by known methods. For example, starting materials represented by formula Rf^{A}—(O)ᵣ—CHF—(CF₂)ₙ—C(O)G′ or [Rf^{B}—(O)ₜ—C(L)H—CF₂—O]ₘ—W—C(O)G′, wherein G′ represents —OH, —O-alkyl (e.g., having from 1 to 4 carbon atoms), or —F and Rf^{A}, Rf^{B}, n, m, L, t, r, and W are as defined above, can be prepared from fluorinated olefins of Formula VI or VII:

Rf^{B}—(O)ₜ—CF═CF₂ VI, or

Rf^{A}—(O)ᵣ—CF═CF₂ VII,

wherein Rf^{A}, Rf^{B}, r, and t are as defined above. Numerous compounds of Formula VI or VII are known (e.g., perfluorinated vinyl ethers and perfluorinated allyl ethers), and many can be obtained from commercial sources (e.g., 3M Company, St. Paul, Minn., and E.I. du Pont de Nemours and Company, Wilmington, Del.). Others can be prepared by known methods; (see, e.g., U.S. Pat. Nos. 5,350,497 (Hung et al.) and 6,255,536 (Worm et al.)).

Compounds of formula Rf^{A}—(O)ᵣ—CHF—(CF₂)ₙ—C(O)G′, wherein n is 0, can be prepared, for example, by reacting a fluorinated olefin of Formula VII with a base (e.g., ammonia, alkali metal hydroxides, and alkaline earth metal hydroxides). Alternatively, for example, a fluorinated olefin of Formula VII can be reacted with an aliphatic alcohol (e.g., methanol, ethanol, n-butanol, and t-butanol) in an alkaline medium, and the resulting ether can be decomposed under acidic conditions to provide a fluorinated carboxylic acid of formula Rf^{A}—(O)ₙ—CHF—(CF₂)ₙ—C(O)G′, wherein n is 0. Compounds of formula Rf^{A}—(O)ᵣ—CHF—(CF₂)ₙ—C(O)G′, wherein n is 1, can be prepared, for example, by a free radical reaction of the fluorinated olefin of Formula VII with methanol followed by an oxidation of the resulting reaction product using conventional methods. Conditions for these reactions are described, for example, in U.S. Pat. App. No. 2007/0015864 (Hintzer et al.), the disclosure of which, relating to the preparation of compounds of formula Rf^{A}—(O)ᵣ—CHF—(CF₂)ₙ—C(O)G′, is incorporated herein by reference. These methods may be useful, for example, for providing structurally pure compounds (e.g., free of other compounds containing other fluorinated segments). In some embodiments, compounds according to the present disclosure are at least 95% (e.g., 96, 97, 98, or 99%) pure.

Fluorinated vinyl ethers of Formulas VI or VII, wherein r and/or t is 1, can be oxidized (e.g., with oxygen) in the presence of a fluoride source (e.g., antimony pentafluoride) to carboxylic acid fluorides of formula Rf^{A}—O—CF₂C(O)F according to the methods described in U.S. Pat. No. 4,987,254 (Schwertfeger et al.), in column 1, line 45 to column 2, line 42, the disclosure of which is incorporated herein by reference. Examples of compounds that can be prepared according to this method include CF₃—(CF₂)₂—O—CF₂—C(O)—CH₃ and CF₃—O—(CF₂)₃—O—CF₂—C(O)—CH₃, which are described in U.S. Pat. No. 2007/0015864 (Hintzer et al.), the disclosure of which, relating to the preparation of these compounds, is incorporated herein by reference. These methods may be useful, for example, for providing structurally pure compounds (e.g., free of other compounds containing other fluorinated segments). In some embodiments, compounds according to the present disclosure are at least 95% (e.g., 96, 97, 98, or 99%) pure.

Compounds of formula [Rf^{b}—(O)ₜ—C(L)H—CF₂—O]ₘ—W—C(O)G′ can be prepared, for example, by reaction of a fluorinated olefin of Formula VI with a hydroxyl compound of Formula VIII according to the reaction:

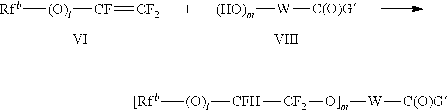

wherein Rf^{b} and t are as defined above, m is 1, 2, or 3, W is alkylene or arylene, and G′ is as defined above. Typically, G′ represents —O-alkyl (e.g., having from 1 to 4 carbon atoms in the alkyl group). Compounds of Formula VIII can be obtained, for example, from commercial sources or can be prepared by known methods. The reaction can be carried out, for example, under conditions described in U.S. Pat. App. No. 2007/0015864 (Hintzer et al.), the disclosure of which, relating to the preparation of compounds of formula [Rf^{b}—(O)ₜ—C(L)H—CF₂—O]ₘ—W—C(O)G′, is incorporated herein by reference.

Fluorinated carboxylic acids and their derivatives according to formula CF₃CFH—O—(CF₂)ₚ—C(O)G′ can be prepared, for example, by decarbonylation of difunctional perfluorinated acid fluoride according to the reaction:

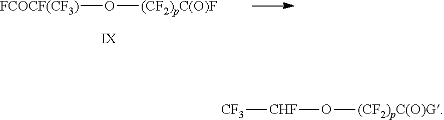

The reaction is typically carried out at an elevated temperature in the presence of water and base (e.g., a metal hydroxide or metal carbonate) according to known methods; see, e.g., U.S. Pat. No. 3,555,100 (Garth et al.), the disclosure of which, relating to the decarbonylation of difunctional acid fluorides, is incorporated herein by reference.

Compounds of Formula IX are available, for example, from the coupling of perfluorinated diacid fluorides of Formula X and hexafluoropropylene oxide according to the reaction:

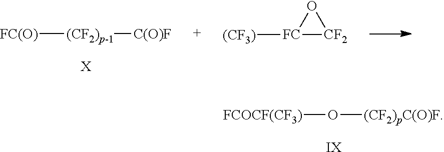

Compounds of Formula X are available, for example, by electrochemical fluorination or direct fluorination of a difunctional ester of formula CH₃OCO(CH₂)ₚ₋₁COOCH₃ or a lactone of formula:

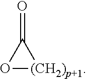

General procedures for carrying out electrochemical fluorination are described, for example, in U.S. Pat. No. 2,713,593 (Brice et al.) and International App. Pub. No. WO 98/50603, published Nov. 12, 1998. General procedures for carrying out direct fluorination are described, for example, in U.S. Pat. No. 5,488,142 (Fall et al.).

Some carboxylic acids and carboxylic acid fluorides useful for preparing compounds represented by formula (Rf-Q)ₐ-X—(Z)_{b}, are commercially available. For example, carboxylic acids of formula CF₃[O—CF₂]₁₋₃C(O)OH are available from Anles Ltd., St. Petersburg, Russia.

Compounds represented by Formula (Rf-Q)ₐ-X—(Z)_{b}, wherein a is 1, can be prepared, for example, from a partially or fully fluorinated carboxylic acid or salt thereof, an acid fluoride thereof, or a carboxylic acid ester (e.g., Rf—C(O)—OCH₃) using a variety of conventional methods. For example, a methyl ester can be treated with an amine having formula NH₂—X—(Z)_{b} according to the following reaction sequence.

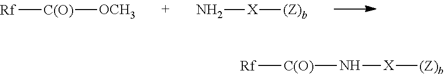

In this sequence, Rf and Z are as defined in any of the above embodiments, X is alkylene, and b is typically 1 or 2. Suitable amines represented by formula NH₂—X—(Z)_{b} include ethanol amine, 3-amino-1-propanol, 2-amino-1-propanol, 4-amino-1-butanol, 3-amino-1,2-propanediol, glycine, iminodiacetic acid, glutamic acid, and aspartic acid. The reaction may be carried out, for example, at an elevated temperature (e.g., up to 80° C., 70° C., 60° C., or 50° C.), and may be carried out neat or in a suitable solvent.

Compounds represented by Formula (Rf-Q)ₐ-X—(Z)_{b}, wherein a and b are each 1, can be prepared, for example, by reducing an ester of formula Rf—C(O)—OCH₃ or a carboxylic acid of formula Rf—C(O)—OH using conventional methods (e.g., by hydride reduction, for example, using sodium borohydride) to a hydroxyl-substituted compound of formula Rf—CH₂OH as shown in the following reaction sequence, wherein Rf is as defined in any of the above embodiments, Q is a bond, and X is CH₂.

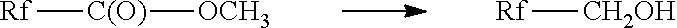

Compounds represented by formula (Rf-Q)ₐ-X—(Z)_{b}, wherein a is greater than 1 (i.e., 2, 3, 4, 5, 6, 7, 8, 9, or 10) and wherein X is an alkylenic polymer backbone can be prepared, for example, by reaction of a partially or fully fluorinated carboxylic acid or salt thereof, an acid fluoride thereof, or a carboxylic acid ester (e.g., Rf—C(O)—OCH₃) using a variety of conventional methods to prepare compounds with polymerizable double bonds, for example, having formula Rf-Q-C(R⁷)═CH₂, which can then be reacted, for example, under free-radical conditions. For example, a compound of formula Rf—(CO)NHCH₂CH₂O(CO)C(R⁷)═CH₂ can be prepared by first reacting Rf—C(O)—OCH₃, for example, with ethanolamine to prepare alcohol-terminated Rf⁴CO)NHCH₂CH₂OH, which can then be reacted, for example, with methacrylic acid, methacrylic anhydride, acrylic acid or acryloyl chloride to prepare the compound of formula Rf⁴CO)NHCH₂CH₂O(CO)C(R⁷)═CH₂, wherein R⁷ is methyl or hydrogen, respectively. Other amino alcohols (e.g., amino alcohols of formula N(R″)HQ″OH) can be used in this reaction sequence to provide compounds of formula Rf—C(O)—N(R″)-Q″-O—C(O)—C(R⁷)═CH₂, wherein Q″ is alkylene or arylalkylene, each of which is optionally interrupted by at least one ether linkage (i.e., —O—), and R″ and R⁷ are as defined above. In another example, Rf—C(O)—OCH₃ can be reacted with allyl amine or N-allyl aniline to prepare a compound of formula Rf—(CO)NHCH₂—CH═CH₂ or Rf—(CO)NH—C₆H₄—CH₂CH₂═CH₂, respectively. Similarly, Rf—C(O)—OCH₃ can be reacted, for example, with allyl alcohol to provide a compound of formula Rf—(CO)OCH₂CH═CH₂. In further examples, an ester of formula Rf—C(O)—OCH₃ or a carboxylic acid of formula Rf—C(O)—OH can be reduced using conventional methods (e.g., hydride, such as sodium borohydride, reduction) to an alcohol of formula Rf—CH₂OH. The alcohol of formula Rf—CH₂OH can then be reacted with methacryloyl chloride, for example, to provide a compound of formula Rf—CH₂O(CO)C(R)═CH₂. The alcohol of formula Rf—CH₂OH can also be reacted with allyl bromide, for example, to provide a compound of formula Rf—CH₂OCH₂CH═CH₂. Examples of suitable reactions and reactants are further disclosed, for example, in the European patent EP 870 778 A1, published Oct. 14, 1998, and U.S. Pat. No. 3,553,179 (Bartlett et al.), the disclosures of which are incorporated herein by reference.

Compounds represented by formula (Rf-Q)ₐ-X—(Z)_{b} and end groups represented by formula (Rf-Q)ₐ-X-(A-)_{b}, wherein b is greater than 1 (i.e., 2, 3, 4, or 5), and wherein X is an alkylenic polymer backbone can be prepared from monomers having a polymerizable double bond and a Z group. For example, a compound formula HO—R⁴—O—C(O)—C(R⁷)═CH₂ may be used, wherein R⁷ is, for example, hydrogen or methyl, and R⁴ is alkylene that is optionally interrupted by at least one ether linkage. Examples of these monomers include hydroxyethyl methacrylate. Other useful monomers include N-methylol acrylamide and isocyanato methacrylate.

Polymeric compounds or end groups represented by formulas (Rf-Q)ₐ-X—(Z)_{b} and (Rf-Q)ₐ-X-(A-)_{b}, respectively, can also be prepared by polymerizing a compound represented by, for example, formula Rf-Q-C(R⁷)═CH₂ and a chain-transfer agent represented by formula HS—R⁶—(Z)_{b}, wherein R⁶ is alkylene, arylene, or arylalkylene, each of which is optionally interrupted by at least one ether linkage. Examples of useful chain transfer agents represented by formula HS—R⁶—(Z)_{b} include 2-mercaptoethanol, mercaptoacetic acid, 2-mercaptobenzoic acid, 3-mercapto-2-butanol, 2-mercaptosulfonic acid, 2-mercaptoethylsulfide, 2-mercaptonicotinic acid, 4-hydroxythiophenol, 3-mercapto-1,2-propanediol, 1-mercapto-2-propanol, 2-mercaptopropionic acid, N-(2-mercaptopropionyl)glycine, 2-mercaptopyridinol, mercaptosuccinic acid, 2,3-dimercaptopropanesulfonic acid, 2,3-dimercaptopropanol, 2,3-dimercaptosuccinic acid, 2,5-dimercapto-1,3,4-thiadiazole, 3,4-toluenedithiol, o-, m-, and p-thiocresol, 2-mercaptoethylamine, ethylcyclohexanedithiol, p-menthane-2,9-dithiol and 1,2-ethanedithiol. In some embodiments, the chain transfer agent is 2-mercaptoethanol or 3-mercapto-1,2-propanediol.

For some embodiments of polymeric compounds or end groups represented by formulas (Rf-Q)ₐ-X—(Z)_{b} and (Rf-Q)ₐ-X-(A-)_{b}, respectively, X comprises at least one (e.g., at least 1, 2, or 5) pendant alkyl ester group. In some of these embodiments, the polymeric compound or end group is preparable by including at least one compound represented by formula:

R⁸—O—C(O)—C(R⁷)═CH₂

in the polymerization reaction to provide at least one divalent unit represented by Formula XI:

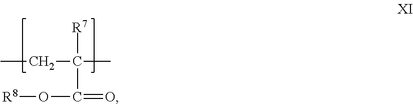

wherein each R⁷ is independently selected from the group consisting of hydrogen and alkyl having from 1 to 4 carbon atoms (e.g., methyl, ethyl, n-propyl, isopropyl, n-butyl, isobutyl, or sec-butyl), and wherein each R⁸ is independently alkyl having from 1 to 30 (in some embodiments, 1 to 25, 1 to 20, 1 to 10, 4 to 25, 8 to 25, or even 12 to 25) carbon atoms. In some embodiments, R⁷ is selected from the group consisting of hydrogen and methyl. In some embodiments, R⁸ is selected from the group consisting of hexadecyl and octadecyl.

Compounds of formula R⁸—O—C(O)—C(R⁷)═CH₂, (e.g., methyl methacrylate, butyl acrylate, hexadecyl methacrylate, octadecyl methacrylate, stearyl acrylate, behenyl methacrylate) are available, for example, from several chemical suppliers (e.g., Sigma-Aldrich Company, St. Louis, Mo.; VWR International, West Chester, Pa.; Monomer-Polymer & Dajac Labs, Festerville, Pa.; Avocado Organics, Ward Hill, Mass.; and Ciba Specialty Chemicals, Basel, Switzerland) or may be synthesized by conventional methods. Some compounds of formula R⁸—O—C(O)—C(R⁷)═CH₂ are available as single isomers (e.g., straight-chain isomer) of single compounds. Other compounds of formula R⁸—O—C(O)—C(R⁷)═CH₂ are available, for example, as mixtures of isomers (e.g., straight-chain and branched isomers), mixtures of compounds (e.g., hexadecyl acrylate and octadecylacrylate), and combinations thereof.

For some embodiments of polymeric compounds or end groups represented by formulas (Rf-Q)ₐ-X—(Z)_{b} and (Rf-Q)ₐ-X-(A-)_{b}, respectively, X comprises at least one (e.g., at least 1, 2, or 5) pendant water-solubilizing group, for example, a polyalkyleneoxy segment, an ammonium group, a carboxylate, a sulfonate, a sulfate, a phosphate, a phosphonate, or an amine-oxide group. In some embodiments, the polymeric compound or end group is preparable by including at least one compound comprising a polyalkyleneoxy segment and represented by formula:

HO-(EO)_{f′}—(PO)_{g′}-(EO)_{f′}—C(O)—C(R⁷)═CH₂;

HO—(PO)_{g′}-(EO)_{f′}—(PO)_{g′}—C(O)—C(R⁷)═CH₂; or

R⁹O-(EO)_{f′}—C(O)—C(R⁷)═CH₂;

in the polymerization reaction to provide a divalent unit represented by formula:

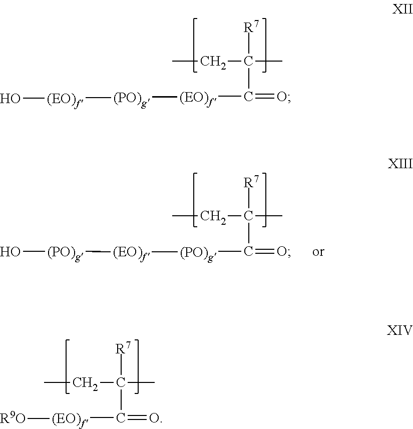

wherein
R⁹ and R⁷ are each independently hydrogen or alkyl of 1 to 4 carbon atoms (e.g., methyl, ethyl, n-propyl, isopropyl, butyl, isobutyl, or t-butyl);
EO represents —CH₂CH₂O—;
each PO independently represents —CH(CH₃)CH₂O— or —CH₂CH(CH₃)O—;
each f′ is independently a number from 1 to 150 (in some embodiments, from 7 to about 140, or from 14 to about 130); and
each g′ is independently a number from 0 to 55 (in some embodiments, from about 21 to about 54 or from about 9 to about 25).

In some embodiments, R⁹ and R⁷ are each independently hydrogen or methyl. In some of these embodiments, the divalent unit is represented by formula:

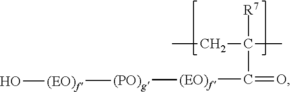

wherein f′ is a number from 5 to 15 (in some embodiments, from 9 to 13 or 11), and
wherein g′ is a number from 15 to 25 (in some embodiments, 19 to 23 or 21).

Compounds of formulas HO-(EO)_{f′}—(PO)_{g′}-(EO)_{f′}—C(O)—C(R⁷)═CH₂, HO—(PO)_{g′}-(EO)_{f′}—(PO)_{g′}—C(O)—C(R⁷)═CH₂, or R⁹O-(EO)_{f′}—C(O)—C(R⁷)═CH₂ can be prepared by known methods, for example, combining acryloyl chloride with a polyethylene glycol having a molecular weight of about 200 to 10,000 grams per mole (e.g., those available from Union Carbide, a wholly owned subsidiary of Dow Chemical, Midland, Mich., under the trade designation “CARBOWAX”) or a block copolymer of ethylene oxide and propylene oxide having a molecular weight of about 500 to 15000 grams per mole (e.g., those available from BASF Corporation, Ludwigshafen, Germany, under the trade designation “PLURONIC”). When a diol-functional copolymer of ethylene oxide and propylene oxide is used, difunctional acrylates (e.g., represented by formula CH₂═C(R⁷)—C(O)—O-(EO)_{f′}—(PO)_{g′}-(EO)_{f′}—C(O)—C(R⁷)═CH₂ or CH₂═C(R⁷)—C(O)—O—(PO)_{g′}-(EO)_{f′}—(PO)_{g′}—C(O)—C(R⁷)═CH₂, wherein f′, g′, R⁷, EO, and PO are as defined above) can be prepared and can be used in a copolymerization reaction with a compound having formula Rf-Q-C(R)═CH₂.

In some embodiments, the polymeric compound or end group represented by formulas (Rf-Q)ₐ-X—(Z)_{b} and (Rf-Q)ₐ-X-(A-)_{b}, respectively, is preparable by including at least one compound represented by formula YOOC—C(R⁷)═CH₂, (YO)₂(O)P—C(R⁷)═CH₂, or Z′—V-Q³C(O)—C(R⁷)═CH₂ in the polymerization reaction to provide an anionic divalent unit represented by formula:

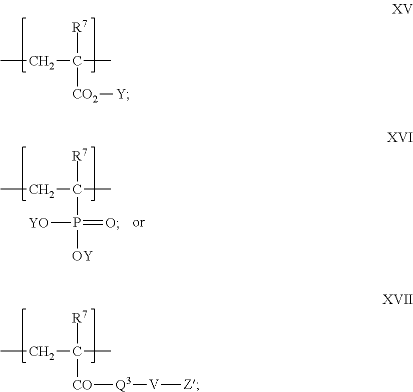

wherein
Q³ is selected from the group consisting of —O—, —S—, and —N(R″)— (in some embodiments, —O—);
R″ and R⁷ are each independently selected from the group consisting of hydrogen and alkyl having from 1 to 4 carbon atoms (e.g., methyl, ethyl, n-propyl, isopropyl, butyl, isobutyl, or t-butyl);
V is alkylene that is optionally interrupted by at least one ether linkage (i.e., —O—) or amine linkage (i.e., —N(R″)—;
each Y is independently selected from the group consisting of hydrogen and a counter cation; and
Z′ is selected from the group consisting of —P(O)(OY)₂, —O—P(O)(O)₂, —SO₃Y, and —CO₂Y.
   In some embodiments, R″ and R⁷ are each independently hydrogen or methyl. In some embodiments, V is alkylene having from 2 to 4 (in some embodiments, 2) carbon atoms. In some embodiments, Y is hydrogen. In some embodiments, Y is a counter cation. Exemplary Y counter cations include alkali metal (e.g., sodium, potassium, and lithium), alkaline earth metal (e.g., calcium and magnesium), ammonium, alkyl ammonium (e.g., tetraalkylammonium), and five to seven membered heterocyclic groups having a positively charged nitrogen atom (e.g., a pyrrolium ion, pyrazolium ion, pyrrolidinium ion, imidazolium ion, triazolium ion, isoxazolium ion, oxazolium ion, thiazolium ion, isothiazolium ion, oxadiazolium ion, oxatriazolium ion, dioxazolium ion, oxathiazolium ion, pyridinium ion, pyridazinium ion, pyrimidinium ion, pyrazinium ion, piperazinium ion, triazinium ion, oxazinium ion, piperidinium ion, oxathiazinium ion, oxadiazinium ion, and morpholinium ion).

Useful compounds represented by formula YOOC—C(R⁷)═CH₂, (YO)₂(O)P—C(R⁷)═CH₂, or Z′—V-Q³C(O)—C(R⁷)═CH₂ include acrylic acid, methacrylic acid, β-carboxyethyl acrylate, β-carboxyethyl methacryate, vinyl phosphonic acid, ethylene glycol methacrylate phosphate, and 2-acrylamido-2-methyl-1-propane sulfonic acid (AMPS).

In some embodiments, the polymeric compound or end group represented by formulas (Rf-Q)ₐ-X—(Z)_{b} and (Rf-Q)ₐ-X-(A-)_{b}, respectively, is preparable by including at least one compound represented by formula Z²—V-Q³C(O)—C(R⁷)═CH₂ in the polymerization reaction to provide a divalent unit represented by formula:

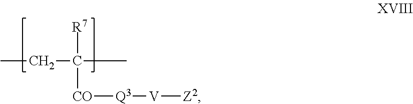

wherein
Q³ is selected from the group consisting of —O—, —S—, and —N(R″)— (in some embodiments, —O—);
R″ and R⁷ are each independently selected from the group consisting of hydrogen and alkyl having from 1 to 4 carbon atoms (e.g., methyl, ethyl, n-propyl, isopropyl, butyl, isobutyl, or t-butyl);
V is alkylene that is optionally interrupted by at least one ether linkage (i.e., —O—) or amine linkage (i.e., —N(R″)— (in some embodiments, alkylene having from 2 to 4 or in some embodiments, 2 carbon atoms); and
Z² is selected from the group consisting of —[N(R¹⁰)₃]⁺E⁻, —N⁺(OY¹)(R¹¹)₃, —N⁺(R¹⁰)₂—(CH₂)₁₋₆—SO₃Y¹, and —N⁺(R¹⁰)₂—(CH₂)₁₋₆—CO₂Y¹, wherein
each R¹⁰ is independently selected from the group consisting of hydrogen and alkyl having from 1 to 6 carbon atoms (e.g., methyl, ethyl, n-propyl, isopropyl, butyl, isobutyl, t-butyl, n-pentyl, isopentyl, n-hexyl);
each R¹¹ is independently selected from the group consisting of hydrogen and alkyl having from 1 to 6 carbon atoms (e.g., methyl, ethyl, n-propyl, isopropyl, butyl, isobutyl, t-butyl, n-pentyl, isopentyl, n-hexyl), wherein alkyl is optionally substituted by at least one halogen, alkoxy, nitro, or nitrile group, or two R¹¹ groups may join to form a 5 to 7-membered ring optionally containing at least one O, N, or S and optionally substituted by alkyl having 1 to 6 carbon atoms;
E⁻ is a counter anion (e.g., acetate, chloride, bromide, iodide, and methylsulphate); and
Y¹ is selected from the group consisting of hydrogen and free anion. In some embodiments, R″ and R⁷ are each independently hydrogen or methyl.

Useful compounds of formula Z²—V-Q³C(O)—C(R⁷)═CH₂ include those that can be prepared from aminoalkyl (meth)acrylates such as N,N-diethylaminoethylmethacrylate, N,N′-dimethylaminoethylmethacrylate and N-t-butylaminoethylmethacrylate, which are commercially available, for example, from Sigma-Aldrich and can be quaternized using conventional techniques, for example, by reaction with an alkyl halide (e.g., bromobutane, bromoheptane, bromodecane, bromododecane, or bromohexadecane) in a suitable solvent and optionally in the presence of a free-radical inhibitor to provide a compound wherein Z² is —[N(R¹⁰)₃]⁺E⁻. Other useful compounds having formula Z²—V-Q³C(O)—C(R⁷)═CH₂ include N,N-dimethylaminoethyl acrylate methyl chloride quaternary and N,N-dimethylaminoethyl methacrylate methyl chloride quaternary available from Ciba Specialty Chemicals, Basel, Switzerland, under the trade designations “CIBA AGEFLEX FA1Q80MC” and “CIBA AGEFLEX FM1Q75MC”, respectively.

For some embodiments of polymeric compounds or end groups represented by formulas (Rf-Q)ₐ-X—(Z)_{b} and (Rf-Q)ₐ-X-(A-)_{b}, respectively, X comprises at least one (e.g., at least 1, 2, or 5) pendant silane group. In some of these embodiments, the polymeric compound or end group is preparable by including at least one compound represented by formula (G)₃-Si—V-Q³C(O)—C(R⁷)═CH₂ in the polymerization reaction to provide a divalent unit represented by formula:

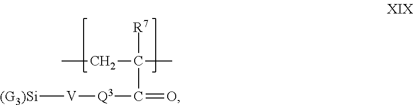

wherein
Q³ is selected from the group consisting of —O—, —S—, and —N(R″)— (in some embodiments, —O—);
R″ and R⁷ are each independently selected from the group consisting of hydrogen and alkyl having from 1 to 4 carbon atoms (e.g., methyl, ethyl, n-propyl, isopropyl, butyl, isobutyl, or t-butyl);
V is alkylene that is optionally interrupted by at least one ether linkage (i.e., —O—) or amine linkage (i.e., —N(R″)— (in some embodiments, alkylene having from 2 to 4 or in some embodiments, 2 carbon atoms); and
each G is independently hydroxyl, alkoxy, acyloxy, aryloxy, halogen, alkyl, or phenyl, wherein at least one (e.g., 2 or 3) G is alkoxy, acyloxy, aryloxy, or halogen.
   In some embodiments, R″ and R⁷ are each independently hydrogen or methyl.

Some compounds of formula [(G)₃Si—V-Q³C(O)—C(R⁷)═CH₂ are commercially available (e.g., CH₂═C(CH₃)C(O)OCH₂CH₂CH₂Si(OCH₃)₃ available, for example, from OSi Specialties, Greenwich, Conn. under the trade designation “SILQUEST A-174 SILANE”).

Silanes can also be incorporated into polymeric compounds or end groups represented by formulas (Rf-Q)ₐ-X—(Z)_{b} and (Rf-Q)ₐ-X-(A-)_{b}, respectively, by using a silane-substituted mercaptan (e.g., 3-mercaptopropyltrimethoxysilane, available, for example, from Huls America, Inc., Somerset, N.J., under the trade designation “DYNASYLAN”) in the polymerization reaction as a chain-tranfer agent.

The polymerization reaction of at least one compound of formula Rf-Q-C(R⁷)═CH₂ and at least one second compound, for example, of formula R⁸—O—C(O)—C(R⁷)═CH₂, HO-(EO)_{f′}—(PO)_{g′}-(EO)_{f′}—C(O)—C(R⁷)═CH₂, HO—(PO)_{g′}-(EO)_{f′}—(PO)_{g′}—C(O)—C(R⁷)═CH₂, R⁹O-(EO)_{f′}—C(O)—C(R⁷)═CH₂, YOOC—C(R⁷)═CH₂, (YO)₂(O)P—C(R⁷)═CH₂, Z′—V-Q³C(O)—C(R⁷)═CH₂, Z²—V-Q³C(O)—C(R⁷)═CH₂, or (G)₃-Si—V-Q³C(O)—C(R⁷)═CH₂ can be carried out in the presence of an added free-radical initiator. Free radical initiators such as those widely known and used in the art may be used to initiate polymerization of the components. Exemplary free-radical initiators are described in U.S. Pat. No. 6,664,354 (Savu et al.), the disclosure of which, relating to free-radical initiators, is incorporated herein by reference. In some embodiments, the polymer or oligomer that is formed is a random graft copolymer. In some embodiments, the polymer or oligomer that is formed is a block copolymer.

In some embodiments, the polymerization reaction is carried out in solvent. The components may be present in the reaction medium at any suitable concentration, (e.g., from about 5 percent to about 80 percent by weight based on the total weight of the reaction mixture). Illustrative examples of suitable solvents include aliphatic and alicyclic hydrocarbons (e.g., hexane, heptane, cyclohexane), aromatic solvents (e.g., benzene, toluene, xylene), ethers (e.g., diethyl ether, glyme, diglyme, and diisopropyl ether), esters (e.g., ethyl acetate and butyl acetate), alcohols (e.g., ethanol and isopropyl alcohol), ketones (e.g., acetone, methyl ethyl ketone and methyl isobutyl ketone), halogenated solvents (e.g., methylchloroform, 1,1,2-trichloro-1,2,2-trifluoroethane, trichloroethylene, trifluorotoluene, and hydrofluoroethers available, for example, from 3M Company, St. Paul, Minn. under the trade designations “HFE-7100” and “HFE-7200”), and mixtures thereof.

Polymerization can be carried out at any temperature suitable for conducting an organic free-radical reaction. Temperature and solvent for a particular use can be selected by those skilled in the art based on considerations such as the solubility of reagents, temperature required for the use of a particular initiator, and desired molecular weight. While it is not practical to enumerate a particular temperature suitable for all initiators and all solvents, generally suitable temperatures are in a range from about 30° C. to about 200° C. (in some embodiments, from about 40° C. to about 100° C., or from about 50° C. to about 80° C.).

Adjusting, for example, the concentration and activity of the initiator, the concentration of each of the reactive monomers, the temperature, the concentration of the chain transfer agent, and the solvent using techniques known in the art can control the molecular weight of a polyacrylate polymer or copolymer.

Compounds according to the present disclosure, in some embodiments, comprise a reaction product of components comprising a multifunctional isocyanate compound. In some of these embodiments, the multifunctional isocyanate compound comprises at least two (e.g., 2, 3, 4, or more) isocyanate groups linked together by alkylene, arylene, or arylalkylene, each of which is optionally attached to at least one of a biuret, an allophanate, an isocyanurate, or a uretdione. In some embodiments, compounds according to the present disclosure comprise a multivalent unit comprising a segment represented by formula:

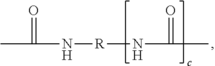

wherein c is 1 to 20 (e.g., 1 to 10, 1 to 6, 1 to 5, 1 to 3, or 1 to 2), and R is alkylene, arylene, or arylalkylene, each of which is optionally interrupted by at least one biruet, allophanate, uretdione, or isocyanurate linkage. Segments represented by this formula may be prepared, for example, by a condensation reaction of a multifunctional isocyanate compound to form carbamate, urea, biuret, or allophanate linkages. In some embodiments, the multifunctional isocyanate is a diisocyanate, wherein two isocyanate (i.e., —NCO) groups are linked by divalent alkylene, arylene, or arylalkylene. In some embodiments, the multifunctional isocyanate is a triisocyanate, wherein three isocyanate groups are independently attached to alkylene, arylene, or arylalkylene groups, which are attached to a biuret or an isocyanurate. Mixtures of multifunctional isocyanate compounds may also be used.

Useful aromatic multifunctional isocyanate compounds include 2,4-toluene diisocyanate (TDI), 2,6-toluene diisocyanate, an adduct of TDI with trimethylolpropane (available, for example, from Bayer Corporation, Pittsburgh, Pa. under the trade designation “DESMODUR CB”), the isocyanurate trimer of TDI (available, for example, from Bayer Corporation under the trade designation “DESMODUR IL”), diphenylmethane 4,4′-diisocyanate (MDI), diphenylmethane 2,4′-diisocyanate, 1,5-diisocyanatonaphthalene, 1,4-phenylene diisocyanate, 1,3-phenylene diisocyanate, 1-methyoxy-2,4-phenylene diisocyanate, 1-chlorophenyl-2,4-diisocyanate, and mixtures thereof.

Useful multifunctional alkylene isocyanate compounds include 1,4-tetramethylene diisocyanate, hexamethylene 1,4-diisocyanate, hexamethylene 1,6-diisocyanate (HDI), 1,12-dodecane diisocyanate, 2,2,4-trimethyl-hexamethylene diisocyanate (TMDI), 2,4,4-trimethyl-hexamethylene diisocyanate, 2-methyl-1,5-pentamethylene diisocyanate, dimer diisocyanate, the urea of hexamethylene diisocyanate, the biuret of hexamethylene 1,6-diisocyanate (HDI) (available, for example, from Bayer Corporation under the trade designations “DESMODUR N-100” and “DESMODUR N-3200”), the isocyanurate of HDI (available, for example, from Bayer Corporation under the trade designations “DESMODUR N-3300” and “DESMODUR N-3600”), a blend of the isocyanurate of HDI and the uretdione of HDI (available, for example, from Bayer Corporation under the trade designation “DESMODUR N-3400”), dicyclohexylmethane diisocyanate (H₁₂ MDI, available, for example, from Bayer Corporation under the trade designation “DESMODUR W”), 4,4′-isopropyl-bis(cyclohexylisocyanate), isophorone diisocyanate (IPDI), cyclobutane-1,3-diisocyanate, cyclohexane 1,3-diisocyanate, cyclohexane 1,4-diisocyanate (CHDI), 1,4-cyclohexanebis(methylene isocyanate) (BDI), 1,3-bis(isocyanatomethyl)cyclohexane (H₆ XDI), 3-isocyanatomethyl-3,5,5-trimethylcyclohexyl isocyanate, and mixtures thereof.

Useful multifunctional arylalkylene isocyanates include m-tetramethyl xylylene diisocyanate (m-TMXDI), p-tetramethyl xylylene diisocyanate (p-TMXDI), 1,4-xylylene diisocyanate (XDI), 1,3-xylylene diisocyanate, p-(1-isocyanatoethyl)-phenyl isocyanate, m-(3-isocyanatobutyl)-phenyl isocyanate, 4-(2-isocyanatocyclohexyl-methyl)-phenyl isocyanate, and mixtures thereof.

In some embodiments of components comprising a multifunctional isocyanate compound, the multifunctional isocyanate compound is hexamethylene 1,6-diisocyanate (HDI), 1,12-dodecane diisocyanate, isophorone diisocyanate, toluene diisocyanate, dicyclohexylmethane 4,4′diisocyanate, diphenylmethane 4,4′-diisocyanate (MDI), the biuret, uretdione, or isocyanurate thereof, and mixtures thereof.

Other useful triisocyanates are those obtained by reacting three moles of a diisocyanate with one mole of a triol. For example, toluene diisocyanate, 3-isocyanatomethyl-3,4,4-trimethylcyclohexyl isocyanate, or m-tetramethylxylene diisocyanate can be reacted with 1,1,1-tris(hydroxymethyl)propane to form triisocyanates. The product from the reaction with m-tetramethylxylene diisocyanate is commercially available, for example, from American Cyanamid, Stamford, Conn. under the trade designation “CYTHANE 3160”.

In some embodiments of compounds comprising a reaction product of components comprising a multifunctional isocyanate and a fluorinated compound, the components further comprise other isocyanate-reactive difunctional or monofunctional materials that can be selected based on the desired application. In some embodiments, the components further comprise at least one of a fluorinated alcohol, fluorinated polyol, a non-fluorinated polyol, an aliphatic alcohol, an aliphatic polyamine, a silane compound represented by formula [(G)₃Si]_{d}—X′—Z, an oxime, a polymerizable compound represented by formula (D)₁₋₃-R³—Z, or a compound represented by formula M-R⁴—Z (e.g., a water-soluble compound), wherein
each G is independently hydroxyl, alkoxy, acyloxy, aryloxy, halogen, alkyl, or phenyl, wherein at least one G is alkoxy, acyloxy, aryloxy, or halogen;
X′ is alkylene or an alkylenic polymer backbone, each of which is optionally interrupted by —S— or —O—, wherein the alkylenic polymer backbone is optionally substituted with at least one alkyl ester group that is optionally substituted with an ammonium group, a carboxylate, a sulfonate, a sulfate, a phosphate, or a phosphonate;
each D is independently acrylate, methacrylate, epoxide, glycidoxy, or vinyl;
M is an ammonium group, a carboxylate, a sulfonate, a sulfate, a phosphate, or a phosphonate;
R³ is divalent, trivalent, or tetravalent alkylene;
R⁴ is alkylene that is optionally interrupted by at least one ether linkage;
and Z is as defined above.

In some embodiments wherein the compound according to the present disclosure is represented by formula:

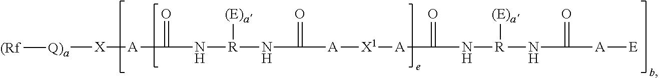

X¹ is alkylene, polyalkyleneoxy, fluoroalkylene, or polyfluoroalkyleneoxy, wherein alkylene is optionally interrupted by at least one of —O—, polydialkylsiloxane, polydiarylsiloxane, or polyalkylarylsiloxane and is optionally substituted with —Si(G)₃, an ammonium group, a polyalkyleneoxy segment, a carboxylate, a sulfonate, a sulfate, a phosphate, or a phosphonate. In some embodiments, X¹ is alkylene or polyalkyleneoxy. In some embodiments, X¹ is fluoroalkylene or polyfluoroalkyleneoxy. In some of these embodiments, each a′ is 0 or 1. In some embodiments, e is a number from 1 to 20 (e.g., 2 to 15 or 3 to 10). In some embodiments, e is 0.

In some embodiments of compounds comprising the reaction product of components comprising a multifunctional isocyanate compound and a fluorinated compound represented by formula (Rf-Q)ₐ-X—(Z)_{b}, the components further comprise a fluorinated polyol. Fluorinated polyols that may be useful in the compounds comprising an reaction product disclosed herein include fluorinated oxetane polyols made by the ring-opening polymerization of fluorinated oxetane (available, for example, from Omnova Solutions, Inc., Akron, Ohio, under the trade designation “POLY-3-FOX”); polyetheralcohols prepared by ring opening addition polymerization of a fluorinated organic group substituted epoxide with a compound containing at least two hydroxyl groups as described in U.S. Pat. No. 4,508,916 (Newell et al); perfluoropolyether diols such as (HOCH₂CF₂O(CF₂O)₈₋₁₂(CF₂CF₂O)₈₋₁₂CF₂CH₂OH, available, for example, from Ausimont, Inc., Thorofare, N.J., under the trade designation “FOMBLIN ZDOL”); 1,4-bis(1-hydroxy-1,1-dihydroperfluoroethoxyethoxy)perfluoro-n-butane (HOCH₂CF₂OC₂F₄O(CF₂)₄OC₂F₄OCF₂CH₂OH); 1,4-bis(1-hydroxy-1,1-dihydroperfluoropropoxy)perfluoro-n-butane (HOCH₂CF₂CF₂O(CF₂)₄OCF₂CF₂CH₂OH), and N-bis(2-hydroxyethyl) perfluorobutylsulfonamide.

In some embodiments of compounds comprising the reaction product of components comprising a multifunctional isocyanate compound and a fluorinated compound represented by formula (Rf-Q)ₐ-X—(Z)_{b}, the components further comprise a non-fluorinated polyol. Non-fluorinated polyols that may be useful in the compounds disclosed herein include alkylene, arylene, arylalkylene, or polymeric groups, which are optionally interrupted with at least one ether linkage (e.g., polyalkyleneoxy compounds) or amine linkage, which have an average hydroxyl functionality of at least about 2 (e.g., up to 5, 4, or 3), and which are optionally substituted with —Si(G)₃, an ammonium group, a carboxylate, a sulfonate, a sulfate, a phosphate, or a phosphonate, wherein each G is independently as defined above. The hydroxyl groups can be primary or secondary. Mixtures of diols with polyols that have a higher average hydroxyl functionality (e.g., 2.5 to 5, 3 to 4, or 3) can also be used. Exemplary non-fluorinated polyols include mono fatty acid esters of polyols (e.g., glycerol monooleate, glycerol monostearate, glycerol monoricinoleate, or C₅ to C₂₀ alkyl di-esters of pentaerythritol); castor oil; polyester diols or polyols (e.g., those available from Union Camp under the trade designation “UNIFLEX”, from Rohm and Haas Co., Philadelphia, Pa. under the trade designation “PARAPLEX U-148”, from Mobay Chemical Corp., Irvine, Calif., under the trade designation “MULTRON”, or those derived from dimer acids or dimer diols and available, for example, from Uniqema, Gouda, Netherlands, under the trade designations “PRIPLAST” or “PRIPOL”; hydroxy-terminated polylactones (e.g., polycaprolactone polyols, for example, with number average molecular weights in the range of about 200 to about 2000 available, for example, from Union Carbide Corp., Danbury, Conn., under the trade designation “TONE”, for example, grades 0201, 0210, 0301, and 0310); hydroxy-terminated polyalkadienes (e.g., hydroxyl-terminated polybutadienes, for example, those available from Elf Atochem, Philadelphia, Pa., under the trade designation “POLY BD”); alkylene diols (e.g., 1,2-ethanediol, 1,2-propanediol, 3-chloro-1,2-propanediol, 1,3-propanediol, 1,3-butanediol, 1,4-butanediol, 2-methyl-1,3-propanediol, 2,2-dimethyl-1,3-propanediol (neopentylglycol), 2-ethyl-1,3-propanediol, 2,2-diethyl-1,3-propanediol, 1,5-pentanediol, 2-ethyl-1,3-pentanediol, 2,2,4-trimethyl-1,3-pentanediol, 3-methyl-1,5-pentanediol, 1,2-, 1,5-, and 1,6-hexanediol, 2-ethyl-1,6-hexanediol, bis(hydroxymethyl)cyclohexane, 1,8-octanediol, bicyclo-octanediol, 1,10-decanediol, tricyclo-decanediol, norbornanediol, and 1,18-dihydroxyoctadecane); polyhydroxyalkanes (e.g., glycerol, trimethylolethane, trimethylolpropane, 2-ethyl-2-(hydroxymethyl)-1,3-propanediol, 1,2,6-hexanetriol, pentaerythritol, quinitol, mannitol, and sorbitol); Bisphenol A ethoxylate, Bisphenol A propyloxylate, and Bisphenol A propoxylate/ethoxylate (available from Sigma-Aldrich, Milwaukee, Wis.); polytetramethylene ether glycols available, for example, from Quaker Oats Company, Chicago, Ill., under the trade designation “POLYMEG”, for example, grades 650 and 1000); polyether polyols available, for example, from E.I. duPont de Nemours, Wilmington, Del., under the trade designation “TERATHANE”); polyoxyalkylene tetrols having secondary hydroxyl groups available, for example, from Wyandotte Chemicals Corporation, Wyandotte, Mich., under the trade designation “PeP”, for example grades 450, 550, and 650; polycarbonate diols (e.g., a hexanediol carbonate with Mₙ=900 available, for example, from PPG Industries, Inc., Pittsburgh, Pa., under the trade designation “DURACARB 120”, aromatic diols (e.g., N,N-bis(hydroxyethyl)benzamide, 4,4′-bis(hydroxymethyl)diphenylsulfone, 1,4-benzenedimethanol, 1,3-bis(2-hydroxyethyoxy)benzene, 1,2-dihydroxybenzene, resorcinol, 1,4-dihydroxybenzene, 3,5-, 2,6-, 2,5-, and 1,6-, 2,6-, 2,5-, and 2,7-dihydroxynaphthalene, 2,2′- and 4,4′-biphenol, 1,8-dihydroxybiphenyl, 2,4-dihydroxy-6-methyl-pyrimidine, 4,6-dihydroxypyrimidine, 3,6-dihydroxypyridazine, bisphenol A, 4,4′-ethylidenebisphenol, 4,4′-isopropylidenebis(2,6-dimethylphenol), bis(4-hydroxyphenyl)methane, 1,1-bis(4-hydroxyphenyl)-1-phenylethane (bisphenol C), 1,4-bis(2-hydroxyethyl)piperazine, bis(4-hydroxyphenyl)ether; and diols and polyols containing another functional group (e.g., bis(hydroxymethyl)propionic acid, 2,4-dihydroxybenzoic acid, N,N-bis(2-hydroxyethyl)-3-aminopropyltriethoxysilane, and bicine).

In some embodiments, the non-fluorinated polyol comprises alkyleneoxy groups, which may be useful, for example, for increasing the water-solubility of the compounds disclosed herein. Useful alkyleneoxy-containing polyols include di and polyalkylene glycols (e.g., di(ethylene glycol), tri(ethylene glycol), tetra(ethylene glycol), dipropylene glycol, diisopropylene glycol, tripropylene glycol, 1,11-(3,6-dioxaundecane)diol, 1,14-(3,6,9,12-tetraoxatetradecane)diol, 1,8-(3,6-dioxa-2,5,8-trimethyloctane)diol, or 1,14-(5,10-dioxatetradecane)diol); polyoxyethylene, polyoxypropylene, and ethylene oxide-terminated polypropylene glycols and triols of molecular weights from about 200 to about 2000 (e.g., available from Union Carbide Corp. under the trade designation “CARBOWAX”, poly(propylene glycol) available, for example, from Lyondell Chemical Company, Houston, Tex., under the trade designation “PPG-425”); and block copolymers of poly(ethylene glycol) and poly(propylene glycol) available from BASF Corporation, Mount Olive, N.J., under the trade designation “PLURONIC”.

Other alkyleneoxy-containing compounds may be useful components in some embodiments for compounds comprising the reaction product of components comprising a multifunctional isocyanate compound and a fluorinated compound represented by formula (Rf-Q)ₐ-X—(Z)_{b}. For example, diamino terminated poly(alkylene oxide) compounds (e.g., those available from Huntsman Corp., The Woodlands, Tex. under the trade designations “JEFFAMINE ED” or “JEFFAMINE EDR-148”) and poly(oxyalkylene) thiols may be used.

In some embodiments of compounds comprising the reaction product of components comprising a multifunctional isocyanate compound, a fluorinated compound, and a non-fluorinated polyol, the non-fluorinated polyol is a polysiloxane diol (e.g., a polydialkylsiloxane diol (e.g., hydroxyalkyl terminated polydimethyl siloxanes, polymethyloctadecylsiloxane, polydimethylmethyloctadecylsiloxane, polydimethyldodecyltetradecylsiloxane, polymethylhexadecylsiloxane, polymethyloctylsiloxane) or polyalkylarylsiloxane diol (e.g., hydroxyalkyl terminated polydiphenylsiloxane or hydroxyalkyl terminated dimethyl-diphenylsiloxane copolymer)) with a polymerization degree, for example, of 5 to 100, 10 to 50, or 10 to 30. In other embodiments, a polysiloxane diamine may be used.

In some embodiments of compounds according to the present disclosure comprising an end group represented by formula (Rf-Q)ₐ-X-(A-)_{b} and a multivalent unit, the compound comprises segments represented by formula:

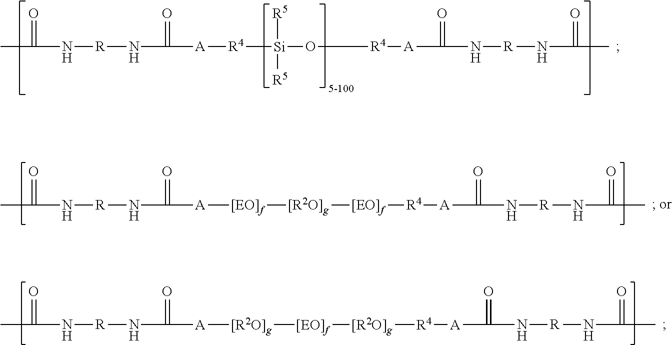

wherein
EO represents —CH₂CH₂O—;
each R²O independently represents —CH(CH₃)CH₂O—, —CH₂CH(CH₃)O—, —CH₂CH₂CH₂O—, —CH(CH₂CH₃)CH₂O—, —CH₂CH(CH₂CH₃)O—, —CH₂CH₂CH₂CH₂O—, or —CH₂C(CH₃)₂O— (in some embodiments, —CH(CH₃)CH₂O— or —CH₂CH(CH₃)O—);
each f is independently a number from 1 to 150 (in some embodiments, from 7 to about 140, or from 14 to about 130);
each g is independently a number from 0 to 55 (in some embodiments, from about 21 to about 54 or from about 9 to about 25);
R⁴ is alkylene that is optionally interrupted by at least one ether linkage or amine linkage (in some embodiments, alkylene having up to 4 carbon atoms); and
each R⁵ is independently alkyl or aryl (e.g., alkyl having up to 4 carbon atoms or phenyl).

In some embodiments of compounds comprising the reaction product of components comprising a multifunctional isocyanate compound and a fluorinated compound represented by formula (Rf-Q)ₐ-X—(Z)_{b}, the components further comprise a monofunctional polyalkyleneoxy compound. In some embodiments, such compounds may have, for example, an end group (e.g., in some embodiments, an E group) represented by formula

alkyl-O-[EO]_{f}—[R²O]_{g}-[EO]_{f}—; or

alkyl-O—[R²O]_{g}-[EO]_{f}—[R²O]_{g}—,

wherein f, g, EO, and R²O are as defined above. In some embodiments, alkyl has up to 4 carbon atoms. Some monofunctional polyalkyleneoxy compounds are commercially available, for example, from Union Carbide under the trade designation “CARBOWAX”.

In some embodiments of compounds comprising the reaction product of components comprising a multifunctional isocyanate compound and a fluorinated compound represented by formula (Rf-Q)ₐ-X—(Z)_{b}, the components further comprise a polyamine. Polyamines that may be useful in the components disclosed herein include alkylene, arylene, arylalkylene, or polymeric groups, which are optionally interrupted with at least one ether linkage (e.g., polyalkyleneoxy compounds) or amine linkage.

In some embodiments of compounds comprising a reaction product of components comprising a multifunctional isocyanate and a fluorinated compound, the components further comprise an aliphatic alcohol, for example, having 1 to 30 (in some embodiments, 4 to 30, 6 to 30, 8 to 25, 10 to 18, or 12 to 16) carbon atoms and one hydroxyl group. Examples of aliphatic alcohols include methanol, ethanol, n-propyl alcohol, isopropyl alcohol, n-butyl alcohol, isobutyl alcohol, t-butyl alcohol, n-amyl alcohol, t-amyl alcohol, 2-ethylhexanol, stearyl alcohol, isostearylalcohol, 1-octanol, 1-decanol, 1-dodecanol, 1-tetradecanol, 1-hexadecanol, and 1-octadecanol. In some embodiments, long-chain hydrocarbon monoalcohols (e.g., those with 8 or more carbon atoms) can be optionally substituted, for example, with groups such as one or more chlorine, bromine, trifluoromethyl, or phenyl groups. In some embodiments, the inclusion of an aliphatic alcohol will result in an end group (e.g., an E group) represented by formula alkyl-A- in a compound disclosed herein, wherein alkyl has 1 to 30 (in some embodiments, 4 to 30, 6 to 30, 8 to 25, 10 to 18, or 12 to 16) carbon atoms, and wherein -A- is —O—.

In some embodiments of compounds comprising a reaction product of components comprising a multifunctional isocyanate and a fluorinated compound, the components further comprise a fluorinated alcohol (i.e., a fluorinated monofunctional alcohol). In some embodiments, the components further comprise a fluorinated monofunctional compound represented by formula Rf³-Q¹-Z, wherein Rf³ is perfluoroalkyl having up to 6 (e.g., 2 to 6 or 4) carbon atoms and optionally interrupted by one or two —O— groups, and wherein Q¹ is alkylene or arylalkylene, wherein alkylene and arylalkylene are optionally interrupted or terminated by at least one functional group that is independently ether, amine, ester, amide, sulfonamide, carbamate, or urea, and Z is as defined above. In some embodiments, Q¹ is alkylene that is optionally terminated on either end by sulfonamide). In some embodiments, Z is —OH. Useful compounds represented by formula Rf³-Q¹-Z include C₄F₉—SO₂NR″-CH₂CH₂OH (e.g., 2-(N-methylperfluorobutanesulfonamido)-ethanol, 2-(N-ethylperfluorobutanesulfonamido)ethanol, or 2-(N-methylperfluorobutane-sulfonamido)propanol), N-methyl-N-(4-hydroxybutyl)perfluorohexanesulfonamide, 1,1,2,2-tetrahydroperfluorooctanol, 1,1-dihydroperfluorooctanol, C₃F₇CON(H)CH₂CH₂OH, 1,1,2,2,3,3-hexahydroperfluorodecanol, C₄F₉—SO₂NR″-CH₂CH₂—O—[CH₂CH₂O]ₜOH, wherein t is 1 to 5, C₄F₉SO₂NR″CH₂CH₂CH₂NH₂, C₄F₉—SO₂NR″-CH₂CH₂SH; C₄F₉—SO₂N—(CH₂CH₂OH)₂, and C₄F₉—SO₂NR″-CH₂CH₂—O—(CH₂)ₛOH wherein s is 2, 3, 4, 6, 8, 10 or 11, wherein R″ is hydrogen or alkyl having up to 4 carbons (e.g., methyl, ethyl and propyl). In some embodiments, compounds disclosed herein comprise an end group (e.g., an E group) represented by formula Rf³-Q¹-A-, wherein Rf³, Q¹, and A are as defined above.

In some embodiments of compounds comprising a reaction product of components comprising a multifunctional isocyanate and a fluorinated compound, the components further comprise a compound represented by formula (M)₁₋₂-R⁴—Z. Each M is independently an ammonium group, a carboxylate (i.e., —CO₂Y), a sulfonate (i.e., —SO₃Y), a sulfate (i.e., —O—SO₃Y or (—O)₂—SO₂Y), phosphate (i.e., —O—P(O)(OY)₂ or (—O)₂—P(O)OY), or a phosphonate (i.e., —P(O)(OY)₂). Ammonium groups include those represented by formula —[N(R¹⁰)₃]⁺E⁻, wherein each R¹⁰ is independently hydrogen, alkyl, or aryl, wherein alkyl and aryl are optionally substituted by at least one halogen, alkoxy, nitro, or nitrile group, and wherein E⁻ is a counter anion, and ring systems having one or two aromatic or saturated rings and a positively charged nitrogen atom (e.g., pyrrolium, pyrimidinium, pyrazolium, isoxazolium, oxazolium, thiazolium, isothiazolium, pyridinium, pyrazinium, pyridazinium, imidazolium, isoindolium, indolium, purinium, quinolinium, isoquinolinium, naphthyridinium, quinoxalinium, quinazolinium, phthalazinium, indazolium, pyrrolidinium, piperidinium, azepinium, or piperazinium). R⁴ is alkylene (e.g., having up to 6 or 4 carbon atoms) that is optionally interrupted by at least one ether linkage or amine linkage, and Z is as defined above. The compounds represented by formula (M)₁₋₂-R⁴—Z when incorporated into the reaction product typically make the reaction product more easily dispersable in water and may enhance its stain release properties. In some embodiments, compounds disclosed herein comprise an end group (e.g., an E group) represented by formula (M)₁₋₂-R⁴-A-, wherein M, R⁴, and A are as defined above.

In some embodiments, Y is hydrogen. In some embodiments, Y is a counter cation. Exemplary Y counter cations include alkali metal (e.g., sodium, potassium, and lithium), alkaline earth metal (e.g., calcium and magnesium), ammonium, alkyl ammonium (e.g., tetraalkylammonium), and five to seven membered heterocyclic groups having a positively charged nitrogen atom (e.g., a pyrrolium ion, pyrazolium ion, pyrrolidinium ion, imidazolium ion, triazolium ion, isoxazolium ion, oxazolium ion, thiazolium ion, isothiazolium ion, oxadiazolium ion, oxatriazolium ion, dioxazolium ion, oxathiazolium ion, pyridinium ion, pyridazinium ion, pyrimidinium ion, pyrazinium ion, piperazinium ion, triazinium ion, oxazinium ion, piperidinium ion, oxathiazinium ion, oxadiazinium ion, and morpholinium ion). Interconversions of Y groups can be carried out, for example, using conventional acid-base chemistry. E⁻ is a counter anion. Typical counter anions include halides (i.e., fluoride, chloride, bromide, and iodide), organic acid salts (e.g., formate, acetate, propionate, lactate, laurate, palmitate, stearate, or citrate), organic sulfonic or sulfuric acid salts (e.g., alkyl sulfates or alkanesulfonates), nitrate, and tetrafluoroborate. The organic acid salts and sulfonic acid salts may be partially fluorinated or perfluorinated. In some embodiments, E⁻ is chloride, bromide, or iodide (i.e., Cl—, Br—, or I—). In some embodiments, E⁻ is selected from the group consisting of chloride, acetate, iodide, bromide, methylsulfate, ethylsulfate, and formate. In some embodiments, E- is a carboxylate.

Exemplary compounds represented by formula (M)₁₋₂-R⁴—Z are glycolic acid (HOCH₂COOH) and its salts, HSCH₂COOH; (HOCH₂CH₂)₂NCH₂COOH, HOC(CO₂H)(CH₂CO₂H)₂, (H₂N(CH₂)ₙCH₂)₂NCH₃ wherein n is a number from 1 to 3, (HOCH₂)₂C(CH₃)COOH; (HO(CH₂)ₙCH₂)₂NCH₃ wherein n is a number from 1 to 3, HOCH₂CH(OH)CO₂Na, N-(2-hydroxyethyl)iminodiacetic acid (HOCH₂CH₂N(CH₂COOH)₂), L-glutamic acid (H₂NCH(COOH)(CH₂CH₂COOH)), aspartic acid (H₂NCH(COOH)(CH₂COOH)), glycine (H₂NCH₂COOH), 1,3-diamino-2-propanol-N,N,N′,N′-tetraacetic acid (HOCH(CH₂N(CH₂COOH)₂)₂), iminodiacetic acid (HN(CH₂COOH)₂), mercaptosuccinic acid (HSCH(COOH)(CH₂COOH)), H₂N(CH₂)₄CH(COOH)N(CH₂COOH)₂, HOCH(COOH)CH(COOH)CH₂COOH, (HOCH₂)₂CHCH₂COO)⁻(NH(CH₃)₃)⁺, CH₃(CH₂)₂CH(OH)CH(OH)(CH₂)₃CO₂K, H₂NCH₂CH₂OSO₃Na, H₂C₂H₄NHC₂H₄SO₃H; H₂C₃H₆NH(CH₃)C₃H₆SO₃H, (HOC₂H₄)₂NC₃H₆OSO₃Na, (HOCH₂CH₂)₂NC₆H₄OCH₂CH₂OSO₂OH, N-methyl-4-(2,3-dihydroxypropoxy)pyridinium chloride, ((H₂N)₂C₆H₃SO₃)⁻(NH(C₂H₅)₃)⁺, dihydroxybenzoic acid, 3,4-dihydroxybenzylic acid, 3-(3,5-dihydroxyphenyl)propionic acid, salts of the above amines, carboxylic acids, and sulfonic acids, and mixtures thereof.

In some embodiments of compounds comprising a reaction product of components comprising a multifunctional isocyanate and a fluorinated compound, the components further comprise an isocyanate blocking agent. Isocyanate blocking agents are compounds that upon reaction with an isocyanate yield a group that is unreactive at room temperature with compounds that are typically isocyanate-reactive at room temperature. Generally, at elevated temperature the blocking group will be released from the blocked (poly)isocyanate compound thereby generating the isocyanate group again, which can then react with an isocyanate-reactive group. Blocking agents and their mechanisms have been described in detail in “Blocked isocyanates III.: Part. A, Mechanisms and Chemistry” by Douglas Wicks and Zeno W. Wicks Jr., Progress in Organic Coatings, 36 (1999), pp. 14-172.

Isocyanate blocking agents include arylalcohols (e.g., phenols), lactams (e.g., ε-caprolactam, δ-valerolactam, and γ-butyrolactam), oximes (e.g., formaldoxime, acetaldoxime, cyclohexanone oxime, acetophenone oxime, benzophenone oxime, 2-butanone oxime, and diethyl glyoxime), bisulfite, and triazoles. In some embodiments, the blocking agent is an oxime. In some of these embodiments, the oxime is represented by formula

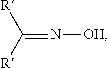

wherein each R′ is independently hydrogen, alkyl (e.g., having up to 4 carbon atoms), or aryl (e.g., phenyl). In some embodiments, compounds disclosed herein comprise an end group (e.g., an E group) represented by formula

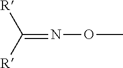

wherein R′ is as defined above.

In some embodiments of compounds comprising a reaction product of components comprising a multifunctional isocyanate and a fluorinated compound, the components further comprise a carbodiimide compound. The carbodiimide compound can be an aromatic or aliphatic carbodiimide compound and may include a polycarbodiimide. Useful carbodiimides include those corresponding to the formula (XX):

R¹²—[N═C═N—R¹⁴]ᵤ—N═C═N—R¹³ (XX)

wherein u has a value of 1 to 20, typically 1 or 2, R¹² and R¹³ each independently represent a hydrocarbon group, in particular a linear, branched or cyclic aliphatic group preferably having 6 to 18 carbon atoms and R¹⁴ represents a divalent linear, branched or cyclic aliphatic group.

The aliphatic carbodiimide extenders of formula XX can be synthesized in a 1-step process by reacting aliphatic diisocyanates (e.g., isophorone diisocyanate, dimer diacid diisocyanate, 4,4′ dicyclohexyl methane diisocyanate) with an aliphatic mono-isocyanate (e.g., n-butyl isocyanate and octadecyl isocyanate) as a chain terminator at 130 to 170° C. in the presence of a phospholine oxide or other suitable carbodiimide formation catalyst (e.g., 1-ethyl-3-phospholine, 1-ethyl-3-methyl-3-phospholine-1-oxide, 3-methyl-1-phenyl-3-phospholine-1-oxide, and bicyclic terpene alkyl or hydrocarbyl aryl phosphine oxide). The reaction is typically carried out in the absence of solvents under inert atmosphere, but high-boiling non-reactive solvents such as methyl isobutyl ketone can be added as diluents. The mole ratio of diisocyanate to mono-isocyanate can be varied from 0.5 to 10, e.g., 1 to 5. A concentration of 0.2 to 5 parts of catalyst per 100 g of diisocyanate is typically suitable. In an alternative approach the aliphatic diisocyanates can be first reacted with monofunctional alcohols, amines or thiols followed by carbodiimide formation in a second step.

In some embodiments of compounds comprising a reaction product of components comprising a multifunctional isocyanate and a fluorinated compound, the components further comprise a silane compound represented by formula [(G)₃Si]_{d}—X′—Z. Each G is independently hydroxyl (i.e., —OH), alkoxy (e.g., —O-alkyl), acyloxy (e.g., —O—C(O)-alkyl), aryloxy (e.g., —O-aryl), oxime (e.g., —O—N═CR′R′) halogen (i.e., fluoride, chloride, bromide, or iodine), alkyl, or phenyl, wherein at least one (in some embodiments, at least two or at least three) G group is alkoxy, acyloxy, aryloxy, or halogen. Alkoxy, acyloxy, aryloxy, or halogen groups are generally capable of hydrolyzing under, for example, acidic or basic aqueous conditions to provide groups (e.g., silanol groups) capable of undergoing condensation reactions (e.g., to form siloxanes or polysiloxanes) and/or reactions with a siliceous or other surface having a metal hydroxide group. In some embodiments, at least one G group is independently alkyl having from 1 to 6 carbon atoms (e.g., methyl, ethyl, n-propyl, isopropyl, n-butyl, isobutyl, sec-butyl, n-pentyl, isopentyl, neopentyl, or n-hexyl). In some embodiments, at least one G group is independently methyl or ethyl. In some embodiments, alkoxy and acyloxy have up to 6 (or up to 4) carbon atoms, and the alkyl group is optionally substituted by halogen. In some embodiments, aryloxy has 6 to 12 (or 6 to 10) carbon atoms which may be unsubstituted or substituted by halogen, alkyl (e.g., having up to 4 carbon atoms), and haloalkyl. In some embodiments, wherein G is an oxime, each R′ is independently hydrogen, alkyl (e.g., having up to 4 carbon atoms), or aryl (e.g., phenyl). In some embodiments, each G is independently selected from the group consisting of halide, hydroxyl, alkoxy, aryloxy, and acyloxy. In some embodiments, each G is independently selected from the group consisting of halide (e.g., chloride) and alkoxy having up to ten carbon atoms. In some embodiments, each G is independently alkoxy having from 1 to 6 (e.g., 1 to 4) carbon atoms. In some embodiments, each G is independently methoxy or ethoxy.

In a silane compound represented by formula [(G)₃Si]_{d}—X′—Z, X′ is alkylene or an alkylenic polymer backbone, each of which is optionally interrupted by —S— or —O—, wherein the alkylenic polymer backbone is optionally substituted with at least one alkyl ester group that is optionally substituted with an ammonium group, a carboxylate, a sulfonate, a sulfate, a phosphate, or a phosphonate. In some embodiments, X′ is alkylene that is optionally interrupted by at least one ether linkage. In some of these embodiments, d is 1. Exemplary X′ groups include —CH₂CH₂—, —CH₂CH₂CH₂—, —CH₂CH₂CH₂OCH₂CH₂—, CH₂CH₂C₆H₄CH₂CH₂—, and —CH₂CH₂O(C₂H₄O)₂CH₂CH₂N(CH₃)CH₂CH₂CH₂—. In some embodiments, X′ is an alkylenic polymer backbone. In these embodiments, d is more than 1 (e.g., 2, 3, 4, or 5). In some embodiments, the alkylenic polymer backbone is substituted with at least one (e.g., at least 2, 3, or 5) alkyl ester group. In some embodiments, the alkylenic polymer backbone is interrupted with —S—. In some embodiments, compounds disclosed herein comprise an end group (e.g., an E group) represented by formula [(G)₃Si]_{d}—X′-A-, wherein G, Si, d, X′, and A are as defined above.

Several silane compounds represented by formula [(G)₃Si]_{d}—X′—Z, wherein d is 1 or 2, are commercially available or are readily preparable using conventional techniques. These compounds include H₂NCH₂CH₂CH₂Si(OC₂H₅)₃, H₂NCH₂CH₂CH₂Si(OCH₃)₃, HN(CH₂CH₂CH₂Si(OCH₃)₃)₂, H₂NCH₂CH₂CH₂Si(O—N═C(CH₃)(C₂H₅))₃, HSCH₂CH₂CH₂Si(OCH₃)₃, HO(C₂H₄O)₃C₂H₄N(CH₃)(CH₂)₃Si(OC₄H₉)₃, H₂NCH₂C₆H₄CH₂CH₂Si(OCH₃)₃, HSCH₂CH₂CH₂Si(OCOCH₃)₃, HN(CH₃)CH₂CH₂Si(OCH₃)₃, HSCH₂CH₂CH₂SiCH₃(OCH₃)₂, (H₃CO)₃SiCH₂CH₂CH₂NHCH₂CH₂CH₂Si(OCH₃)₃, HN(CH₃)C₃H₆Si(OCH₃)₃, CH₃CH₂OOCCH₂CH(COOCH₂CH₃)HNC₃H₆Si(OCH₂CH₃)₃, C₆H₅NHC₃H₆Si(OCH₃)₃, H₂C₃H₆SiCH₃(OCH₂CH₃)₂, HOCH(CH₃)CH₂OCONHC₃H₆Si(OCH₂CH₃)₃, (HOCH₂CH₂)₂NCH₂CH₂CH₂Si(OCH₂CH₃)₃, 3-isocyanatopropyltriethoxysilane, 3-isocyanatopropyltrimethoxysilane, and mixtures thereof. Polymeric silanes can be prepared, for example, by free-radical polymerization under the conditions, for example, described previously for the preparation of polymeric fluorinated compounds represented by formula (Rf-Q)ₐ-X—(Z)_{b}.

In some embodiments of compounds comprising a reaction product of components comprising a multifunctional isocyanate and a fluorinated compound, the components further comprise a polymerizable compound represented by formula (D)₁₋₃-R³—Z. Each D is independently acrylate, methacrylate, epoxide, glycidoxy, or vinyl. In some embodiments, each D is independently acrylate or methacrylate. R³ is divalent, trivalent, or tetravalent alkylene. Representative compounds represented by formula (D)₁₋₃-R³—Z, which are commercially available or readily synthesized by conventional techniques, include pentaerylthritol triacrylate, hydroxyethyl acrylate, hydroxyethyl methacrylate, glycidol, allyl alcohol, and 1,4-butanediol vinyl ether. In some embodiments, compounds disclosed herein comprise an end group (e.g., an E group) represented by formula (D)₁₋₃-R³-A-, wherein D, R³, and A are as defined above.

Compounds according to the present disclosure can be prepared, for example, by a condensation reaction between a fluorinated compound represented by formula (Rf-Q)ₐ-X—(Z)_{b} and a multifunctional isocyanate compound, optionally containing other components as described above. The conditions for carrying out such condensation reactions are known in the art. Typically, the reaction is run in the presence of a catalyst, for example, a tin II or tin IV salt (e.g., dibutyltin dilaurate, stannous octanoate, stannous oleate, tin dibutyldi-(2-ethyl hexanoate), tin (II) 2-ethyl hexanoate, and stannous chloride) or a tertiary amine (e.g., triethylamine, tributylamine, triethylenediamine, tripropylamine, bis(dimethylaminoethyl)ether, ethyl morpholine, 2,2′-dimorpholinodiethyl ether, 1,4-diazabicyclo[2.2.2]octane (DABCO), and 1,8-diazabicyclo[5.4.0.]undec-7-ene (DBU). In some embodiments, a tin salt is used. The amount of catalyst present will depend on the particular reaction. Generally, however, suitable catalyst concentrations are from about 0.001 percent to about 10 percent (in some embodiments, about 0.1 percent to about 5 percent or about 0.1 to about 1 percent) by weight based on the total weight of the reactants. Typically, the reaction will be carried out such that all or almost all (e.g., greater than 90, 95, 98, 99, or 99.5 percent) isocyanate groups have been reacted, resulting in a product that is essentially free of isocyanate groups.

The condensation reaction useful for the preparation of compounds according to the present disclosure is typically carried out under dry conditions in common, non-protic organic solvents (e.g., ethyl acetate, acetone, methyl isobutyl ketone, methyl ethyl ketone, and toluene) and fluorinated solvents (e.g., hydrofluoroethers and trifluorotoluene). Suitable reaction temperatures can be determined by those skilled in the art based on the particular reagents, solvents, and catalysts being used. Generally suitable reaction temperatures are between about room temperature and about 120° C. (e.g., 30° C. to 100° C., 40° C. to 90° C., or 60° C. to 80° C.). Generally the reaction is carried out such that between 1 and 100 percent (e.g., from 5 to 60, 10 to 50, or 10 to 40 percent) of the isocyanate groups of the multifunctional isocyanate compound or mixture of multifunctional isocyanate compounds is reacted with the fluorinated compound represented by formula (Rf-Q)ₐ-X—(Z)_{b}. The remainder of the isocyanate groups is reacted with one or more of the components described above. For example, an oligomeric compound may be obtained by reacting 10 to 30 percent of the isocyanate groups with the fluorinated compound represented by formula (Rf-Q)ₐ-X—(Z)_{b}, reacting 90 to 30 percent of the isocyanate groups with an isocyanate blocking agent, and reacting 0 to 40 percent of the isocyanate groups with water or a fluorinated alcohol, an aliphatic alcohol, a silane compound represented by formula [(G)₃Si]_{d}—X′—Z, a polymerizable compound represented by formula (D)₁₋₃-R³—Z, or a compound represented by formula (M)₁₋₂-R⁴—Z. When a compound represented by formula (M)₁₋₂-R⁴—Z is used, typically the ratio of the multifunctional isocyanate compound to the compound represented by formula (M)₁₋₂-R⁴—Z is from about 3:1 to about 16:1 (e.g., 5:1 to about 11:1). The order of the addition of components can be changed as would be understood by a person of skill in the art.

In some embodiments, the multifunctional isocyanate compound is combined with a fluorinated or non-fluorinated polyol in addition to the fluorinated compound represented by formula (Rf-Q)ₐ-X—(Z)_{b}. A mixture of polyols can be used instead of a single polyol. When the multifunctional isocyanate compound is a triisocyanate, the polyol is typically a diol to prevent undesired gelation. The resulting isocyanate functional oligomers are then further reacted with a fluorinated compound represented by formula (Rf-Q)ₐ-X—(Z)_{b} and at least one of a fluorinated alcohol, an aliphatic alcohol, a silane compound represented by formula [(G)₃Si]_{d}—X′—Z, a polymerizable compound represented by formula (D)₁₋₃-R³—Z, or a compound represented by formula (M)₁₋₂-R⁴—Z. End groups represented by formula (Rf-Q)ₐ-X-(A-)_{b} are thereby bonded to the isocyanate functional oligomers. In some embodiments, a compound represented by formula [(G)₃Si]_{d}—X′—Z (e.g., an aminosilane) is used in the reaction mixture.

In some embodiments, the present disclosure provides an oligomer represented by formula RfQ-X—O(—CONH—R(R¹⁶)ₘ—NHCO—OR¹⁵O—)ₙCONH—R(R¹⁶)—NHCO-AX′Si(G)₃ or RfQ-X—O(—CONH—R(R¹⁶)ₘ—NHCO—OR¹⁵O—)ₙCONHX′Si(G)₃, wherein Rf, Q, X, R, A, X′, and G are as defined in any of the above embodiments; R¹⁵ is a divalent organic group which is derived from the polyol and may be substituted with water-solubilizing groups (e.g., carboxylate, sulfate, sulfonate, phosphonate, ammonium, and mixtures thereof) and may be substituted with fluorinated groups; and R¹⁶ is Rf-Q-X—OCONH—, (G)₃SiX′A-CONH—, or M₁₋₂-R⁴—CONH—, wherein Rf, Q, X, G, X′, A, M, and R⁴ are as defined above; m is a number from 0 to 2; and n, which is the number of repeating units, is a number from 2 to 10. In some embodiments, the oligomer has a weight average molecular weight not more than 100,000 grams per mole or 50,000 grams per mole (e.g., in a range from 1500 to 15,000 grams per mole or from 1500 to 5,000 grams per mole). Typically, the oligomeric compound has a molecular weight such that it is readily dissolved or dispersed in water or an organic solvent.

Compounds according to the present disclosure can be dispersed or dissolved in water or organic solvent for use in the methods disclosed herein comprising treating a surface. The term “dispersed” as used herein includes dispersions of a solid in a liquid as well as liquid in liquid dispersions (i.e., emulsions). The resulting dispersion or solution (e.g., for treating a surface) typically includes from at least 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.5, 1, 1.5, 2, 3, 4, or 5 percent by weight, up to 5, 6, 7, 8, 9, or 10 percent by weight of at least one fluorinated compound according to the present disclosure, based on the total weight of the solution or dispersion. For example, the amount of a fluorinated compound according to the present disclosure in a solution or dispersion may be in a range of from 0.01 to 10, 0.1 to 10, 0.1 to 5, 1 to 10, or from 1 to 5 percent by weight, based on the total weight of the solution or dispersion. Lower and higher amounts of the compound in the solution or dispersion may also be used, and may be desirable for some applications. The ratio of the solvents, water and optionally other components may be chosen to provide a homogeneous mixture.

A compound according to the present disclosure may be stored in the form of a concentrate (e.g., a concentrated solution of a compound disclosed herein in organic solvent). The concentrate may be stable for several weeks (e.g., at least one, two, or three months). The compound according to the present disclosure may be present in the concentrate in an amount of at least 10, 20, 25, 30, 40, 50, 60, or at least 70 percent by weight, based on the total weight of the concentrate. Typically, the compound disclosed herein may be present in the concentrate in an amount ranging from 10 percent and 50 percent by weight. Concentrates may be diluted shortly before use (e.g., before application to a surface), for example, with water, organic solvent, and optionally acid or base.

When the compound disclosed herein is in the form of a dispersion in water or an organic solvent, the weight average particle size of the particles of the compound may be up to 400 nm (e.g., up to 300 nm).

In some embodiments, the compound disclosed herein is formulated into an aqueous dispersion. The dispersion may be stabilized using non-fluorinated surfactants (e.g., polyoxyalkylene surfactants or polyoxyethylene surfactants such as those available from Clariant under the trade designation “EMULSOGEN EPN 207” and from Uniqema under the trade designation “TWEEN 80”); anionic non-fluorinated surfactants (e.g., lauryl sulfate and sodium dodecyl benzene sulfonate); cationic non-fluorinated surfactants (e.g., those available from Akzo under the trade designations “ARQUAD T-50” and “ETHOQUAD 18-25”); and zwitterionic non-fluorinated surfactants (e.g., lauryl amineoxide and cocamido propyl betaine). The non-fluorinated surfactant may be present in an amount of about 1 to about 25 parts by weight (e.g., 2 to about 10 parts by weight), based on 100 parts by weight of the compound disclosed herein.

In embodiments wherein a compound according to the present disclosure contains at least one of a carboxylate, a sulfonate, a sulfate, a phosphate, or a phosphonate, the compound can be converted into a salt (i.e., wherein Y is a counter cation) before or after the compound is dispersed or dissolved in water. In some embodiments, a salt forming compound is added in a water phase after all of the isocyanate groups have been reacted. Useful salt forming compounds include ammonia, tertiary amines (e.g., trimethylamine, triethylamine, tripropylamine, triisopropylamine, tributylamine, triethanolamine, diethanolamine, methyldiethanolamine, morpholine, N-methylmorpholine, dimethylethanolamine, and mixtures thereof), quaternary ammonium hydroxides, and inorganic bases (e.g., sodium hydroxide, potassium hydroxide, cesium hydroxide, lithium hydroxide, calcium hydroxide, magnesium hydroxide, zinc hydroxide, and barium hydroxide). The salt forming compounds may be used, for example, in an amount to maintain a pH of greater than about 6.

In some embodiments, the compound disclosed herein is formulated into a solution or dispersion in organic solvent (e.g., one or more organic solvents). The term solvent refers to a liquid material or a mixture of liquid materials that is capable of at least partially dissolving a compound disclosed herein at 25° C. In some embodiments, the solvent is capable of dissolving at least 0.01% by weight of the compound disclosed herein. In some embodiments, the solvent is capable of dissolving at least 0.1% by weight water. Suitable organic solvents include aliphatic alcohols (e.g., methanol, ethanol, isopropyl alcohol, or t-butanol), ketones (e.g., acetone, isobutyl methyl ketone, or methyl ethyl ketone), esters (e.g., ethyl acetate, butyl acetate, or methylformate), ethers (e.g., diisopropyl ether), and ether-alcohols (methoxy propanol). Fluorinated solvents may be used in combination with the organic solvents. Examples of fluorinated solvents include fluorinated hydrocarbons (e.g., perfluorohexane or perfluorooctane), partially fluorinated hydrocarbons (e.g., pentafluorobutane or CF₃CFHCFHCF₂CF₃), and hydrofluoroethers, (e.g., methyl perfluorobutyl ether, ethyl perfluorobutyl ether, or hydrofluoroethers available, for example, from 3M Company, St. Paul, Minn., under the trade designations “HFE-7100” or “HFE-7200”). In some of these embodiments, the solution or dispersion further comprises water (e.g., in an amount between 0.1 and 20 (e.g., 0.5 to 15 or 1 to 10) percent by weight based on the total weight of the solution or dispersion).

Formulations containing compounds according to the present disclosure may contain further additives such as buffering agent, agents to impart fire proofing or antistatic properties, fungicidal agents, optical bleaching agents, sequestering agents, mineral salts, and swelling agents to promote penetration.

Formulations containing compounds according to the present disclosure, in embodiments wherein the compound comprises an end group represented by formula [(G)₃Si]_{d}—X′-A-, may also contain a compound represented by formula (G)₄M′, wherein M′ is Si, Ti, Zr, or Al, and wherein each G is independently hydroxyl, alkoxy, acyloxy, aryloxy, halogen, alkyl, or phenyl, wherein at least one G is alkoxy, acyloxy, aryloxy, or halogen. Representative compounds of this formula include tetramethoxysilane, tetraethoxysilane, methyltriethoxysilane, dimethyldiethoxysilane, octadecyltriethoxysilane, methyltrichlorosilane, tetramethyl orthotitanate, tetraethyl orthotitanate, tetraisopropyl orthotitanate, tetraethylzirconate, tetraisopropylzirconate, and tetrapropylzirconate. The weight ratio of a compound represented by formula (G)₄M′ and a compound according to the present disclosure may be, for example, in a range from 3:1 to 12:1, or in a range from 6:1 to 9:1. In some embodiments wherein the compound comprises an end group represented by formula [(G)₃Si]_{d}—X′-A-, useful formulations comprise one of an acid or a base. The acid may be an organic or inorganic acid. Organic acids include acetic acid, citric acid, formic acid, and fluorinated organic acids, such as CF₃SO₃H, C₃F₇COOH, C₇F₁₅COOH, C₆F₁₃P(O)(OH)₂, or a fluorinated organic acid represented by the Formula R_{f}⁹—[—(Y)ⱼ—Z]ₖ, wherein R_{f}⁹ represents a mono or divalent polyfluoropolyether group, Y represents an organic divalent linking group, Z represents an acid group (e.g., a carboxylic acid group), j is 0 or 1, and k is 1 or 2. Exemplary fluorinated organic acids represented by formula R_{f}⁹—[—(Y)ⱼ—Z]ₖ include C₃F₇O(CF(CF₃)CF₂O)₁₀₋₃₀CF(CF₃)COOH (commercially available from E.I. DuPont de Nemours and Company, Wilmington, Del., under the trade designations “KRYTOX 157 FSH”, “KRYTOX 157 FSL”, and “KRYTOX 157 FSM”) and CF₃(CF₂)₂OCF(CF₃)COOH. Examples of inorganic acids include sulfuric acid, hydrochloric acid, and phosphoric acid. In some embodiments, the acid is at least one of acetic acid, citric acid, formic acid, para-toluenesulfonic acid, triflic acid, perfluorobutyric acid, hydroboric acid, sulfuric acid, phosphoric acid, or hydrochloric acid. Useful bases include amines (e.g., triethylamine), alkali metal hydroxides (e.g., sodium hydroxide or potassium hydroxide), alkaline earth metal hydroxides, or ammonium hydroxide. The acid or base will generally be included in the formulation in an amount between about 0.005 and 10% (e.g., between 0.01 and 10% or between 0.05 and 5%) by weight, based on the total weight of the formulation.

In some embodiments of methods of making an article having a surface and articles according to the present disclosure, the article is a fibrous material (e.g., fabric, textiles, carpets, leather, and paper). The fibrous material may be woven or nonwoven and may contain synthetic fibers (e.g., polyester, polyamide and polyacrylate fibers), natural fibers (e.g., cellulose fibers), and mixtures thereof.

Treating the surface of the article can be carried out, for example, by immersing the article in a formulation comprising a compound disclosed herein or by spraying the article with such a formulation. The treated article can then be run through a padder/roller to remove excess formulation and subsequently dried. The treated article may be dried at room temperature by leaving it in air, may be subjected to a heat treatment (e.g., in an oven), or both. The heat treatment may be carried out at temperatures between about 50° C. and about 190° C. (e.g., 120° C. to 170° C. or about 150° C. to about 170° C.) for a period of about 20 seconds to 10 minutes (e.g., 3 to 5 minutes).

Typically, compounds according to the present disclosure can be applied to fibrous articles in a range from 0.05% to 3% by weight (e.g., 0.2 to 1% by weight) based on the weight of the fibrous article. The amount of the compound applied to the fibrous article is chosen to maximize the desired properties without substantially affecting the look and feel of the treated substrate.

In some embodiments of methods and articles according to the present disclosure, a hard surface is treated. Useful surfaces include ceramics, glazed ceramics, glass, metal, natural and man-made stone, thermoplastic materials (e.g., poly(meth)acrylate, polycarbonate, polystyrene, styrene copolymers (e.g., styrene acrylonitrile copolymers), polyesters, or polyethylene terephthalate), paints (such as those based on acrylic resins), powder coatings (such as polyurethane or hybrid powder coatings), and wood. In some embodiments, the surface comprises functional groups capable of reacting with the fluorinated compound according to the present disclosure. Such reactivity of the surface may occur naturally (e.g., in a siliceous surface), or a reactive surface may be provided by treatment in a plasma containing oxygen or in a corona atmosphere.

Various articles can be treated with a fluorinated compound according to the present disclosure to provide a water- and oil-repellent coating thereon. Exemplary articles include ceramic tiles, bathtubs, sinks, toilet bowls, glass shower panels, construction glass, various parts of a vehicle (e.g. mirror or windows), ceramic or enamel pottery materials, lenses used in ophthalmic spectacles, sunglasses, optical instruments, illuminators, watch crystals, plastic window glazing, signs, decorative surfaces such as wallpaper and vinyl flooring, composite or laminated substrates (e.g., sheeting available from Formica Corporation, Cincinnati, Ohio under the trade designation “FORMICA” and flooring available, for example, from Pergo, Raleigh, N.C. under the trade designation “PERGO”), natural and man-made stones, decorative and paving stones (e.g., marble, granite, limestone, and slate), cement and stone sidewalks and driveways, particles that comprise grout or the finished surface of applied grout, wood furniture surface (e.g., desktops and tabletops), cabinet surfaces, wood flooring, decking, and fencing, leather, paper, fiber glass fabric and other fiber-containing fabrics, textiles, carpeting, kitchen and bathroom faucets, taps, handles, spouts, sinks, drains, hand rails, towel holders, curtain rods, dish washer panels, refrigerator panels, stove tops, panels on stoves, ovens, or microwaves, exhaust hoods, grills, and metal wheels or rims.

In some embodiments, the surface of the article to be treated may be cleaned before treatment so that it is substantially free of organic contamination. Cleaning techniques depend on the type of substrate and include a solvent washing step with an organic solvent (e.g., acetone or ethanol).

A wide variety methods can be used to treat a hard surface with a compound disclosed herein (e.g., brushing, spraying, dipping, rolling, or spreading). An article can typically be treated with a compound at room temperature (typically, about 20° C. to about 25° C.). Alternatively, the mixture can be applied to substrates that are preheated (e.g., at a temperature of 60° C. to 150° C. This may be useful, for example, in industrial production of, for example, ceramic tiles, which can be treated immediately after exiting the baking oven at the end of the production line. Following application, the treated substrate can be dried and cured at ambient or elevated temperature (e.g., from 40° to 300° C., 50° C. to 190° C., 120° C. to 170° C., or about 150° C. to about 170° C.) for a period of about 20 seconds to 10 minutes (e.g., 3 to 5 minutes). In some embodiments, methods disclosed herein further comprise a polishing step to remove excess material. Compounds disclosed herein are generally applied to a surface in amounts sufficient to produce a coating which is water- and oil-repellent. This coating can be extremely thin (e.g. 10 to 200 nanometers) or, in some applications, may be thicker.

For either the treatment of fibrous substrates or hard substrates, the heating step may be useful, for example, to deblock blocked isocyanate groups (e.g., oxime-blocked isocyanates). The deblocked isocyanates may then react, for example, with each other, with water, or with the substrate. For embodiments in which the compounds disclosed herein comprise an end group represented by formula [(G)₃Si]_{d}—X′-A-, heating may cause hydrolysis of the G groups (e.g., alkoxy, acyloxy, or halogen), which typically generates silanol groups that can participate in condensation reactions to form siloxanes and/or participate in bonding interactions with silanol groups or other metal hydroxide groups on the surface of articles treated according to the present disclosure. The bonding interaction may be through a covalent bond (e.g., through a condensation reaction) or through hydrogen bonding. Hydrolysis can occur, for example, in the presence of water optionally in the presence of an acid or base. At neutral pH, hydrolysis typically takes place at 40° C. to 200° C. or 50° C. to 100° C. The water necessary for hydrolysis may be added to the formulation containing the compound that is used to treat the article, may be adsorbed to the surface of the article, or may be present in the atmosphere to which the fluorinated compound is exposed (e.g., an atmosphere having a relative humidity of at least 10%, 20%, 30%, 40%, or even at least 50%).

Compounds according to the present disclosure that have an end group represented by formula (D)₁₋₃-R³—Z may be included in formulations that have a catalyst for the polymerization of the D group. For example, a photoinitiator or other free-radical initiator may be incorporated into a formulation comprising a compound wherein D is an acrylate or methacrylate group. The methods of making an article having a surface may further comprise exposing the formulation to uv light to initiate the polymerization of the acrylate or methacrylate group.

The compounds disclosed herein, which have partially fluorinated polyether groups and/or have fully fluorinated polyether groups with a low number (e.g., up to 4) continuous perfluorinated carbon atoms, are herein demonstrated to have useful water- and oil-repellent properties and may provide a lower-cost alternative to repellents having a larger number of continuous perfluorinated carbon atoms.

In some embodiments, methods of making an article having a surface according to the present disclosure increase the contact angle of a surface to at least one of water or hexadecane. In some embodiments, the methods provide a treated surface having at a contact angle at 20° C. with distilled water of at least 80°, 85°, 90°, 95°, or at least 100°, measured after the treatment has been heated. In some embodiments, the methods provide a treated surface having at a contact angle at 20° C. with n-hexadecane of at least 40°, 45°, 50°, 55°, or at least 60° measured after the treatment has been heated.

In some embodiments, treating the surface with the compound provides a contact angle of at least one of water or hexadecane on the surface that is higher than a contact angle provided by treating an equivalent surface with a comparative compound, wherein the comparative compound is the same as the compound except that the end group is replaced by a comparative end group represented by formula: C₃F₇—O—CF(CF₃)—C(O)—NH—CH₂CH₂—O—. The term “equivalent surface” refers to a surface that is the same in all respects except for the identity of the surface treatment.

In one aspect, the present disclosure provides a method of reducing the surface tension of a liquid, the method comprising combining the liquid with an amount of a compound disclosed herein, wherein the amount of the compound is sufficient to reduce the surface tension of the liquid. In some of these embodiments, the liquid is water. In some of these embodiments, the water is part of an aqueous coating formulation. These aqueous formulations may be useful, for example, for coatings (e.g., floor finishes, varnishes, automotive coatings, marine coatings, sealers, hard coats for plastic lenses, coatings for metal cans or coils, and inks). When used in aqueous formulations (e.g., for coatings) compounds according to the present invention can be formulated into an aqueous solution or dispersion at a final concentration, for example, of about 0.001 to about 1 weight percent (wt. %), about 0.001 to about 0.5 wt. %, or about 0.01 to about 0.3 wt. %, based on the weight of the solution or dispersion. In some embodiments, compounds according to the present disclosure (e.g., example, which contain a polyalkyleneoxy segment or a water-solubilizing group M) may enhance wetting and/or leveling of a coating (e.g., an aqueous coating) on a substrate surface and may provide better dispersability of a component (e.g., a thickening agent or pigment) within the coating formulation. In some embodiments, the coating formulation may include a non-fluorinated polymer.

### EXEMPLARY EMBODIMENTS

1. A compound comprising:
an end group represented by formula:

(Rf-Q)ₐ-X-(A-)_{b}; and
a multivalent unit comprising a segment represented by formula:

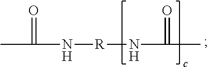

wherein
each Rf is independently:

Rf^{A}—(O)ᵣ—CHL′-(CF₂)ₙ—;

[Rf^{B}—(O)ₜ—C(L)H—CF₂—O]ₘ—W—;

CF₃CFH—O—(CF₂)ₚ—;

CF₃—(O—CF₂)_{z}—; or

CF₃—O—(CF₂)₃—O—CF₂—;
each Q is independently alkylene or arylalkylene, wherein alkylene and arylalkylene are optionally interrupted or terminated by at least one functional group that is independently ether, amine, ester, amide, carbamate, or urea, wherein when “a” is 1, Q may also be a bond, —C(O)O—, or —C(O)—N(R″)—;
X is alkylene or an alkylenic polymer backbone, each of which is optionally interrupted by —S— or —O—, wherein the alkylenic polymer backbone is optionally substituted with at least one alkyl ester group that is optionally substituted with —Si(G)₃, an ammonium group, a polyalkyleneoxy segment, a carboxylate, a sulfonate, a sulfate, a phosphate, or a phosphonate, wherein each G is independently hydroxyl, alkoxy, acyloxy, aryloxy, halogen, alkyl, or phenyl, and wherein at least one G is alkoxy, acyloxy, aryloxy, or halogen;
each A is independently —O—, —N(R¹)—, —S—, or —C(O)O—;
R″ and R¹ are each independently hydrogen or alkyl having up to 4 carbon atoms;
a is a number from 1 to 10;
b is a number from 1 to 5;
c is a number from 1 to 20;
Rf^{A} and Rf^{B} independently represent a partially or fully fluorinated alkyl group having from 1 to 6 carbon atoms and optionally interrupted with at least one oxygen atom;
L is F or CF₃;
L′ is F or H;
W is alkylene or arylene;
r is 0 or 1, wherein when r is 0, then Rf^{A} is interrupted with at least one oxygen atom;
t is 0 or 1;
m is 1, 2, or 3;
n is 0 or 1;
each p is independently a number from 1 to 6;
z is a number from 2 to 7; and
R is alkylene, arylene, or arylalkylene, each of which is optionally interrupted by at least one biruet, allophanate, uretdione, or isocyanurate linkage.
   2. The compound according to embodiment 1, wherein the compound further comprises an end group represented by formula:

alkyl-A-;

Rf³-Q¹-A-;

alkyl-O-[EO]_{f}-[R²O]_{g}-[EO]_{f}—;

alkyl-O—[R²O]_{g}-[EO]_{f}—[R²O]_{g}—;

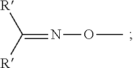

[(G)₃Si]_{d}—X′-A-;

(D)₁₋₃-R³-A-; or

(M)₁₋₂-R⁴-A-;

wherein
Rf³ is perfluoroalkyl having up to 6 carbon atoms and optionally interrupted by one or two —O— groups;
Q¹ is alkylene or arylalkylene, wherein alkylene and arylalkylene are optionally interrupted or terminated by at least one functional group that is independently ether, amine, ester, amide, sulfonamide, carbamate, or urea;
EO represents —CH₂CH₂O—;
each R²O independently represents —CH(CH₃)CH₂O—, —CH₂CH(CH₃)O—, —CH₂CH₂CH₂O—, —CH(CH₂CH₃)CH₂O—, —CH₂CH(CH₂CH₃)O—, —CH₂CH₂CH₂CH₂O—, or —CH₂C(CH₃)₂O—;
each f is independently a number from 1 to 150;
each g is independently a number from 0 to 55;
each G is independently hydroxyl, alkoxy, acyloxy, aryloxy, halogen, alkyl, or phenyl, wherein at least one G is alkoxy, acyloxy, aryloxy, or halogen;
each D is independently acrylate, methacrylate, epoxide, glycidoxy, or vinyl;
M is an ammonium group, a carboxylate, a sulfonate, a sulfate, a phosphate, or a phosphonate;
each R′ is independently hydrogen, alkyl, or aryl;
R³ is divalent, trivalent, or tetravalent alkylene;
R⁴ is alkylene that is optionally interrupted by at least one ether linkage or amine linkage;
X′ is alkylene or an alkylenic polymer backbone, each of which is optionally interrupted by —S— or —O—, wherein the alkylenic polymer backbone is optionally substituted with at least one alkyl ester group that is optionally substituted with an ammonium group, a carboxylate, a sulfonate, a sulfate, a phosphate, or a phosphonate; and
d is a number from 1 to 5.
   3. The compound according to embodiment 1, wherein the compound is represented by formula:

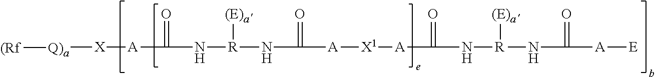

wherein

each a′ is independently 0, 1, or 2;

e is a number from 0 to 20;

X¹ is alkylene, polyalkyleneoxy, fluoroalkylene, or polyfluoroalkyleneoxy, wherein alkylene is optionally interrupted by at least one of —O—, polydialkylsiloxane, polydiarylsiloxane, or polyalkylarylsiloxane and is optionally substituted with —Si(G)₃, an ammonium group, a polyalkyleneoxy segment, a carboxylate, a sulfonate, a sulfate, a phosphate, or a phosphonate;

each E is independently an end group represented by formula:

(Rf-Q)ₐ-X-A-;

alkyl-A-;

Rf³-Q¹-A-;

alkyl-O-[EO]_{f}-[R²O]_{g}-[EO]_{f}—;

alkyl-O—[R²O]_{g}-[EO]_{f}-[R²O]_{g}—;

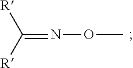

[(G)₃Si]_{d}—X′-A-;

(D)₁₋₃-R³-A-; or

(M)₁₋₂-R⁴-A-; wherein
Rf³ is perfluoroalkyl having up to 6 carbon atoms and optionally interrupted by one or two —O— groups;
Q¹ is alkylene or arylalkylene, wherein alkylene and arylalkylene are optionally interrupted or terminated by at least one functional group that is independently ether, amine, ester, amide, sulfonamide, carbamate, or urea;
EO represents —CH₂CH₂O—;
each R²O independently represents —CH(CH₃)CH₂O—, —CH₂CH(CH₃)O—, —CH₂CH₂CH₂O—, —CH(CH₂CH₃)CH₂O—, —CH₂CH(CH₂CH₃)O—, —CH₂CH₂CH₂CH₂O—, or —CH₂C(CH₃)₂O—;
each f is independently a number from 1 to 150;
each g is independently a number from 0 to 55;
each G is independently hydroxyl, alkoxy, acyloxy, aryloxy, halogen, alkyl, or phenyl, wherein at least one G is alkoxy, acyloxy, aryloxy, or halogen;
each D is independently acrylate, methacrylate, epoxide, glycidoxy, or vinyl;
M is an ammonium group, a carboxylate, a sulfonate, a sulfate, a phosphate, or a phosphonate;
each R′ is independently hydrogen, alkyl, or aryl;
R³ is divalent, trivalent, or tetravalent alkylene;
R⁴ is alkylene that is optionally interrupted by at least one ether linkage or amine linkage;
X′ is alkylene or an alkylenic polymer backbone, each of which is optionally interrupted by —S— or —O—, wherein the alkylenic polymer backbone is optionally substituted with at least one alkyl ester group that is optionally substituted with an ammonium group, a carboxylate, a sulfonate, a sulfate, a phosphate, or a phosphonate; and
d is a number from 1 to 5.
   4. The compound according to any preceding embodiment, wherein the compound comprises segments represented by formula:

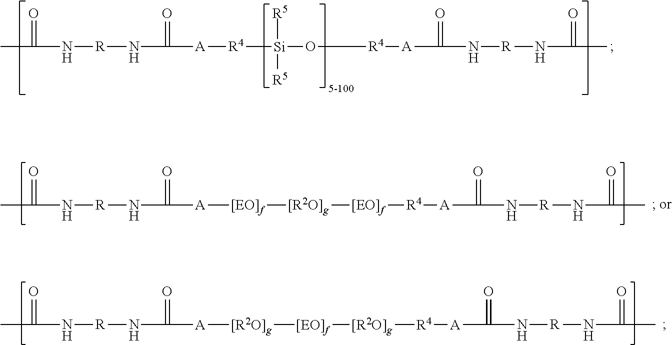

wherein
EO represents —CH₂CH₂O—;
each R²O independently represents —CH(CH₃)CH₂O—, —CH₂CH(CH₃)O—, —CH₂CH₂CH₂O—, —CH(CH₂CH₃)CH₂O—, —CH₂CH(CH₂CH₃)O—, —CH₂CH₂CH₂CH₂O—, or —CH₂C(CH₃)₂O—;
each f is independently a number from 1 to 150;
each g is independently a number from 0 to 55;
R⁴ is alkylene that is optionally interrupted by at least one ether linkage or amine linkage; and
each R⁵ is independently alkyl or aryl.
   5. A compound comprising a reaction product of components comprising a multifunctional isocyanate compound and a fluorinated compound represented by formula:

(Rf-Q)ₐ-X—(Z)_{b}

wherein
each Rf is independently:

Rf^{A}—(O)ᵣ—CHL′-(CF₂)ₙ—;

[Rf^{B}—(O)ₜ—C(L)H—CF₂—O]ₘ—W—;

CF₃CFH—O—(CF₂)ₚ—;

CF₃—(O—CF₂)_{z}—; or

CF₃O—(CF₂)₃—O—CF₂—;
each Q is independently alkylene or arylalkylene, wherein alkylene and arylalkylene are optionally interrupted or terminated by at least one functional group that is independently ether, amine, ester, amide, carbamate, or urea, wherein when “a” is 1, Q may also be a bond, —C(O)O—, or —C(O)—N(R″)—, and wherein R″ is hydrogen or alkyl having up to 4 carbon atoms;
X is alkylene or an alkylenic polymer backbone, each of which is optionally interrupted by —S— or —O—, wherein the alkylenic polymer backbone is optionally substituted with at least one alkyl ester group that is optionally substituted with —Si(G)₃, an ammonium group, a polyalkyleneoxy segment, a carboxylate, a sulfonate, a sulfate, a phosphate, or a phosphonate, wherein each G is independently hydroxyl, alkoxy, acyloxy, aryloxy, halogen, alkyl, or phenyl, and wherein at least one G is alkoxy, acyloxy, aryloxy, or halogen;
each Z is independently hydroxyl, amino, mercaptan, isocyanate, epoxy, or a carboxylic acid;
a is a number from 1 to 10;
b is a number from 1 to 5;
Rf^{A} and Rf^{B} independently represent a partially or fully fluorinated alkyl group having from 1 to 6 carbon atoms and optionally interrupted with at least one oxygen atom;
L is F or CF₃;
L′ is F or H;
W alkylene or arylene;
r is 0 or 1, wherein when r is 0, then Rf^{A} is interrupted with at least one oxygen atom;
t is 0 or 1;
m is 1, 2, or 3;
n is 0 or 1;
each p is independently a number from 1 to 6; and
z is a number from 2 to 7.
   6. The compound according to embodiment 5, wherein the components further comprise at least one of a fluorinated alcohol, fluorinated polyol, a non-fluorinated polyol, an aliphatic alcohol, a polyamine, a silane compound represented by formula [(G)₃Si]_{d}—X′—Z, an oxime, a polymerizable compound represented by formula (D)₁₋₃-R³—Z, or a compound represented by formula (M)₁₋₂-R⁴—Z, wherein
each G is independently hydroxyl, alkoxy, acyloxy, aryloxy, halogen, alkyl, or phenyl, wherein at least one G is alkoxy, acyloxy, aryloxy, or halogen;
X′ is alkylene or an alkylenic polymer backbone, each of which is optionally interrupted by —S— or —O—, wherein the alkylenic polymer backbone is optionally substituted with at least one alkyl ester group that is optionally substituted with an ammonium group, a carboxylate, a sulfonate, a sulfate, a phosphate, or a phosphonate;
each D is independently acrylate, methacrylate, epoxide, glycidoxy, or vinyl;
each M is independently an ammonium group, a carboxylate, a sulfonate, a sulfate, a phosphate, or a phosphonate;
R³ is divalent, trivalent, or tetravalent alkylene; and
R⁴ is alkylene that is optionally interrupted by at least one ether linkage.
   7. The compound according to embodiment 5 or 6, wherein the multifunctional isocyanate compound comprises at least two isocyanate groups linked together by alkylene, arylene, or arylalkylene, each of which is optionally attached to at least one of a biuret, an allophanate, an isocyanurate, or a uretdione.
   8. The compound according to any preceding embodiment, wherein each Rf is independently:

Rf^{A}—(O)ᵣ—CHF—(CF₂)ₙ—;

[Rf^{B}—(O)ₜ—C(L)H—CF₂—O]ₘ—W—; or

CF₃CFH—O—(CF₂)ₚ—.

9. The compound according to any preceding embodiment, wherein t and r are each 1, and wherein Rf^{A} and Rf^{B} are independently selected from the group consisting of:

fully fluorinated alkyl groups having from 1 to 3 carbon atoms; and

fully fluorinated groups represented by formula:

R_{f}¹—[OR_{f}²]ₓ—,

wherein
R_{f}¹ is a perfluorinated alkyl group having from 1 to 3 carbon atoms;
each R_{f}² is independently perfluorinated alkylene having from 1 to 3 carbon atoms; and
x is a value from 1 to 4.
   10. The compound according to any one of embodiments 1 to 8, wherein t and r are each 0, and wherein Rf^{A} and Rf^{B} are independently a fully fluorinated group represented by formula:

R_{f}⁴—[OR_{f}⁵]_{y}—O—CF₂—,

wherein
R_{f}⁴ is a perfluorinated alkyl group having from 1 to 3 carbon atoms;
each R_{f}⁵ is independently perfluorinated alkylene having from 1 to 3 carbon atoms; and
y is a value from 0 to 4.
   11. The compound according to any of embodiments 1 to 8, wherein Rf is:

C₃F₇—O—CHF—;

CF₃—O—CF₂CF₂—CF₂—O—CHF—;

CF₃—O—CF₂—CF₂—O—CHF—;

CF₃—O—CF₂—O—CF₂—CF₂—O—CHF—;

CF₃—O—CHF—CF₂—;

CF₃—O—CF₂—CF₂—O—CHF—CF₂—;

CF₃—CF₂—O—CHF—CF₂—;

CF₃—O—CF₂—CF₂—CF₂—O—CHF—CF₂—;

CF₃—O—CF₂—O—CF₂—CF₂—O—CHF—CF₂—;

CF₃—O—CF₂—CHF—;

C₃F₇—O—CF₂—CHF—;

CF₃—O—CF₂—CF₂—CF₂—O—CF₂—CHF—;

CF₃—O—CF₂—O—CF₂—CF₂—O—CF₂—CHF—;

CF₃—O—CF₂—CHF—CF₂—;

C₂F₅—O—CF₂—CHF—CF₂—;

C₃F₇—O—CF₂—CHF—CF₂—;

CF₃—O—CF₂—CF₂—CF₂—O—CF₂—CHF—CF₂—; or

CF₃—O—CF₂—O—CF₂—CF₂—O—CF₂—CHF—CF₂—.

12. The fluorinated compound according to embodiment 11, wherein Rf is CF₃—O—CF₂CF₂—CF₂—O—CHF—; CF₃—O—CF₂—CF₂—CF₂—O—CF₂—CHF—; CF₃—O—CF₂—CF₂—CF₂—O—CHF—CF₂—; or CF₃—O—CF₂—CF₂—CF₂—O—CF₂—CHF—CF₂—.
13. The compound according to any of embodiments 1 to 8, wherein Rf is:

CF₃—O—CHF—CF₂—O—CH₂—;

CF₃—O—CF₂—CF₂—CF₂—O—CHF—CF₂—O—CH₂—;

C₃F₇—O—CHF—CF₂—O—CH₂—;

C₃F₇—O—CHF—CF₂—O—CH₂—CH₂—;

C₃F₇—O—CF₂—CHF—CF₂—OCH₂—;

CF₃—CHF—CF₂—O—CH₂—; or

C₃F₇—CF₂—CHF—CF₂—OCH₂—.

14. The compound according to any of embodiments 1 to 8, wherein Rf is CF₃CFH—O—(CF₂)ₚ—, and wherein p is 3 or 5.
15. The compound according to any of embodiments 1 to 7, wherein Rf is CF₃—(O—CF₂)_{z}—, and wherein z is 3 or 4.
16. The compound according to any of embodiments 1 to 7, wherein Rf is CF₃—O—(CF₂)₃—O—CF₂—.
17. The compound according to any preceding embodiment, wherein X is an alkylenic polymer backbone.
18. The compound according to embodiment 17, wherein (Rf-Q)ₐ-X— is represented by formula:

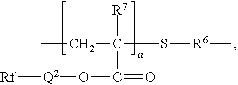

wherein
each Q² is independently —C(O)—N(R″)-alkylene, alkylene, —C(O)—N(R″)-arylalkylene, or arylalkylene, each of which is optionally interrupted by at least one ether linkage and wherein R″ is hydrogen or alkyl having up to 4 carbon atoms;
R⁶ is alkylene, arylene, or arylalkylene, each of which is divalent or trivalent and optionally interrupted by at least one ether linkage; and
each R⁷ is independently hydrogen or methyl.
   19. The compound according to any of embodiments 1 to 16, wherein a and b are each 1, wherein X is alkylene, and wherein Q is a bond, —C(O)O—, or —C(O)—N(R″)—.
   20. A method of making an article having a surface, the method comprising treating the surface with a compound according to any preceding embodiment.
   21. An article comprising a surface, wherein at least a portion of the surface is treated with a compound according to any one of embodiments 1 to 19.
   22. The method according to embodiment 20, wherein treating the surface with the compound provides a contact angle of at least one of water or hexadecane on the surface that is higher than a contact angle provided by treating an equivalent surface with a comparative compound, wherein the comparative compound is the same as the compound except that the end group is replaced by a comparative end group represented by formula:

C₃F₇—O—CF(CF₃)—C(O)—NH—CH₂CH₂—O—.

23. A method of reducing the surface tension of a liquid, the method comprising combining the liquid with an amount of a compound according to any of embodiments 1 to 19, wherein the amount of the compound is sufficient to reduce the surface tension of the liquid.

Embodiments of this disclosure are further illustrated by the following non-limiting examples, but the particular materials and amounts thereof recited in these examples, as well as other conditions and, details, should not be construed to unduly limit this disclosure.

### EXAMPLES

In the following examples, all reagents were obtained from Sigma-Aldrich, St. Louis, Mo., or Bornem, Belgium unless indicated otherwise. All percentages and ratios reported are by weight unless indicated otherwise.

### Example 1

### Part A

The methyl ester of perfluoro-3,7-dioxaoctanoic acid (CF₃OCF₂CF₂CF₂OCF₂C(O)OCH₃) was prepared according to the method described in U.S. Pat. App. Pub. No. 2007/0015864 (Hintzer et al.) in the Preparation of Compound 1, the disclosure of which preparation is incorporated herein by reference.

### Part B

In a three-necked 100-mL flask fitted with a stirrer, thermometer, and condenser were placed 18 grams (0.05 mole) of CF₃OCF₂CF₂CF₂OCF₂C(O)OCH₃, prepared in Part A, and ethanolamine (3.1 grams, 0.05 mole) under a nitrogen atmosphere. The reaction mixture was heated under nitrogen at 50° C. using a heating mantle for two hours. Methanol was then removed under reduced pressure. A clear, yellow, slightly viscous liquid was obtained, which was identified to be CF₃OCF₂CF₂CF₂OCF₂C(O)NHCH₂CH₂OH using NMR and IR spectroscopy.

### Part C

In a three-necked 100-mL flask fitted with a stirrer, thermometer, and condenser were placed 3.9 grams (0.01 mole) of CF₃OCF₂CF₂CF₂OCF₂C(O)NHCH₂CH₂OH, prepared in Part B, 6 grams (0.03 isocyanate equivalents) of the biuret of hexamethylene-1,6-diisocyanate obtained from Bayer Material Science, Pittsburgh, Pa., under the trade designation “DESMODUR N-100”, 10 grams of methyl ethyl ketone, and 1 drop of tin(II) 2-ethylhexanoate. The reaction mixture was heated with a heating mantle to 80° C. under nitrogen atmosphere, stirred for 6 hours, and then cooled to 50° C. under nitrogen. Pentaerythritoltriacrylate (PETA) (10.1 grams, 0.02 hydroxyl equivalents) and 1 spoontip each of 4-methoxyphenol (MEHQ) and phenothiazine were then added. The reaction mixture was stirred for 16 hours at 80° C. A clear, amber solution resulted. Analysis by IR spectroscopy indicated no residual isocyanate was left.

### Example 2

Example 2 was prepared according to the method of Example 1, except CF₃OCF₂OCF₂OCF₂OCF₂COOCH₃ (4.2 grams, 0.01 mole) was used in Part B instead of CF₃OCF₂CF₂CF₂OCF₂C(O)OCH₃.

CF₃OCF₂OCF₂OCF₂OCF₂COOCH₃ was prepared by esterification of perfluoro-3,5,7,9-tetraoxadecanoic acid (obtained from Anles Ltd., St. Petersburg, Russia) in methanol using 50% aqueous sulfuric acid. Flash distillation of the reaction mixture resulted in a two-phase distillate. The lower phase was fractionally distilled to provide the methyl ester of perfluoro-3,5,7,9-tetraoxadecanoic acid.

### Example 3

Example 3 was prepared according to the method of Example 1, except (3-aminopropyl)trimethoxysilane (APTMS) (3.7 grams) was used instead of PETA, MEHQ, and phenothiazine in Part C.

### Example 4

Example 4 was prepared according to the method of Example 1, except CF₃OCF₂OCF₂OCF₂OCF₂COOCH₃ (4.2 grams, 0.01 mole, prepared as described in Example 2) was used in Part B instead of CF₃OCF₂CF₂CF₂OCF₂C(O)OCH₃ and APTMS (3.7 grams) was used instead of PETA, MEHQ, and phenothiazine in Part C.

### Example 5

### Part A

The methyl ester of 3-H-perfluoro-4,8-dioxanonanoic acid (CF₃O(CF₂)₃OCHFCF₂COOCH₃) was prepared according to the method described in the synthesis of compound 2 in U.S. Pat. App. Pub. No. 2007/0142541 (Hintzer et al.); the disclosure of this synthesis is incorporated herein by reference.

### Part B

CF₃O(CF₂)₃OCHFCF₂COOCH₃ (19.6 grams, 0.05 mole) was treated according to the method of Example 1, Part B to provide CF₃OCF₂CF₂CF₂OCHFCF₂C(O)NHCH₂CH₂OH, which was identified using NMR and IR spectroscopy.

### Part C

In a three-necked 100-mL flask fitted with a stirrer, thermometer, and condenser were placed 3.7 grams (0.01 mole) of CF₃OCF₂CF₂CF₂OCHFCF₂C(O)NHCH₂CH₂OH, prepared in Part B, 2.2 grams (0.01 mole) of isophoronediisocyanate (obtained from Aldrich, Bornem, Belgium), 8 grams of methyl ethyl ketone, and 1 drop of tin(II) 2-ethylhexanoate. The reaction mixture was heated with a heating mantle to 80° C. under nitrogen atmosphere, stirred for 4 hours, and then cooled to 50° C. under nitrogen. Stearyl alcohol (2.7 grams, 0.01 mole) was then added. The reaction mixture was stirred for 16 hours at 80° C. to provide a clear, amber solution. Analysis by IR spectroscopy indicated no residual isocyanate was left.

### Example 6

Example 6 was prepared according to the method of Example 5, except 2-butanoneoxime (1.8 grams) was used instead of stearyl alcohol in Part C.

### Example 7

Example 7 was prepared according to the method of Example 5, except 7.5 grams of a monofunctional methoxypolyethyleneglycol with a molecular weight of 750 grams per mole (obtained from Dow Chemical, Midland, Mich., under the trade designation “CARBOWAX 750”) was used instead of stearyl alcohol in Part C.

### Example 8

Example 8 was prepared according to the method of Example 5, except CF₃OCF₂OCF₂OCF₂OCF₂COOCH₃ (4.2 grams, 0.01 mole, prepared as described in Example 2) was used in Part B instead of CF₃O(CF₂)₃OCHFCF₂COOCH₃, 6 grams of the biuret of hexamethylene 1,6-diisocyanate obtained from Bayer Material Science, Pittsburgh, Pa., under the trade designation “DESMODUR N-100” was used instead of isophorone diisocyanate in Part C, and 2-butanoneoxime (1.8 grams) was used instead of stearyl alcohol in Part C.

### Example 9

Example 9 was prepared according to the method of Example 5, except CF₃OCF₂OCF₂OCF₂OCF₂COOCH₃ (4.2 grams, 0.01 mole, prepared as described in Example 2) was used in Part B instead of CF₃O(CF₂)₃OCHFCF₂COOCH₃ and 3.5 grams of a monofunctional methoxypolyethyleneglycol with a molecular weight of 350 grams per mole (obtained from Dow Chemical, Midland, Mich., under the trade designation “CARBOWAX 350”) was used instead of stearyl alcohol in Part C.

### Example 10

Example 10 was prepared according to the method of Example 5, except CF₃OCF₂OCF₂OCF₂OCF₂COOCH₃ (4.2 grams, 0.01 mole, prepared as described in Example 2) was used in Part B instead of CF₃O(CF₂)₃OCHFCF₂COOCH₃ and 1.3 grams of 2-ethylhexanol was used instead of stearyl alcohol in Part C.

### Example 11

Example 11 was prepared according to the method of Example 5, except CF₃OCF₂OCF₂OCF₂OCF₂COOCH₃ (4.2 grams, 0.01 mole, prepared as described in Example 2) was used in Part B instead of CF₃O(CF₂)₃OCHFCF₂COOCH₃ and 5 grams of a bis(hydroxypropyl) terminated polydimethylsiloxane with a molecular weight of 500 grams per mole (obtained from Shin-Etsu, Tokyo, Japan, under the trade designation “X-22 160AS”) was used instead of stearyl alcohol in Part C.

### Example 12

Example 12 was prepared according to the method of Example 5, except CF₃OCF₂OCF₂OCF₂OCF₂COOCH₃ (4.2 grams, 0.01 mole, prepared as described in Example 2) was used in Part B instead of CF₃O(CF₂)₃OCHFCF₂COOCH₃ and 22 grams of a block copolymer of ethylene oxide and propylene oxide (obtained from BASF Corporation, Ludwigshafen, Germany, under the trade designation “PLURONIC L44”) was used instead of stearyl alcohol in Part C.

### Example 13

### Part A

In a three-necked 500-mL flask fitted with a stirrer, thermometer, and condenser were placed 0.1 mole of CF₃OCF₂CF₂CF₂OCF₂C(O)NHCH₂CH₂OH, prepared in Example 1, Part B, 60 grams methyl ethyl ketone, 60 grams of a hydrofluoroether obtained from 3M Company, St. Paul, Minn. under the trade designation “HFE-7200”, 0.1 mole (10.1 grams) of triethylamine, 0.01 grams MEHQ and 0.01 grams phenothazine. The mixture was cooled to about 5° C. in an ice bath. Then 0.11 mole acryloylchloride (10.1 grams) was added dropwise over about 1 hour under nitrogen. An exothermic reaction was noticed, and precipitate formed. The temperature was allowed to rise to 25° C. over a period of about 1 hour while the reaction mixture was stirred. The stirring was continued for 1 hour under nitrogen at 50° C. The resulting reaction mixture was washed 3 times with 200 mL of water and the organic layer was separated off. All solvents were distilled of at 50° C. under vacuum. A clear, yellow-brown liquid was obtained, which was identified to be CF₃OCF₂CF₂CF₂OCF₂C(O)NHCH₂CH₂OC(O)CH═CH₂ using nuclear magnetic resonance spectroscopy.

### Part B

In a three-necked 100-mL flask fitted with a thermometer, stirrer, and condenser were placed 4.4 grams (0.01 mole) of the material from Part A, 0.2 gram (0.0025 mole) 2-mercaptoethanol, and 0.05 gram 2,2′-azobis(2-methylpropionitrile) (AIBN) in 5 grams ethyl acetate. The reaction mixture was degassed 3 times using nitrogen and aspirator vacuum and then heated to 75° C. for 6 hours. Another charge of 0.02 gram AIBN was added and the reaction was continued for 16 hours at 75° C. under a nitrogen atmosphere to provide a 48% solution of hydroxyl-terminated oligomer.

### Part C

In a three-necked 500-mL flask fitted with a stirrer, thermometer, and condenser were placed 9.6 grams (containing 0.0025 mole of hydroxyl-terminated oligomer) of the solution from Part B, 1.5 gram (0.0075 isocyanate equivalents) of the biuret of hexamethylene 1,6-diisocyanate obtained from Bayer Material Science under the trade designation “DESMODUR N-100”, and 1 drop of tin(II) 2-ethylhexanoate. The reaction mixture was heated with a heating mantle to 80° C. under nitrogen atmosphere, stirred for 16 hours, and then cooled to about 30° C. under nitrogen. 2-Butanoneoxime (0.4 gram, 0.005 mole) was added, and the reaction mixture was stirred for 3 hours at about 50° C. to provide a clear, slightly yellow solution. Analysis by IR spectroscopy indicated no residual isocyanate was left.

### Comparative Example A

Comparative Example A was prepared according to the method of Example 1 except 17.2 grams (0.05 mole) of CF₃CF₂CF₂OCF(CF₃)COOCH₃ (obtained, and formerly available, from Hoechst AG, Germany as the methyl ester of perfluoro-(beta-propoxy)-propionic acid) instead of CF₃OCF₂CF₂CF₂OCF₂C(O)OCH₃ in Part B, and APTMS (10.1 grams of 99% pure material, 0.05 mole) was used instead of was used instead of PETA, MEHQ, and phenothiazine in Part C.

### Dynamic Contact Angle on Glass

The solutions of Examples 1 to 13 were diluted to 1% by weight with methyl ethyl ketone. Flat glass substrates (obtained from Aqua Production, France) were dip-coated in each of the resulting solutions and allowed to dry at room temperature. Examples 1 and 2 were passed six times under a 200 W/inch lamp (253 nm, obtained from American Ultraviolet Company, Murray Hill, N.J.) at 20 feet per minute. Examples 3 to 13 were heated for 3 minutes at 120° C. in an oven. Dynamic advancing and receding contact angles were measured using a Kruss DSA 100 (obtained from Kruss GmbH, Hamburg, Germany). The results, which represent the average of 3 measurements, are summarized in Table 1, below.

For Comparative Example B, a solution containing 10 grams of a 10% by weight fluorinated disilane solution (obtained from 3M Company, St. Paul, Minn., under the trade designation “3M EASY CLEAN COATING ECC-4000”), 10 grams of 37% hydrochloric acid, and 980 grams ethanol was prepared. The solution was spray applied to flat glass. The pressure during spraying was about 2 bar (2×10⁵ Pa), the flow about 40 mL/minute, and the add-on about 150 mL/m². Each substrate was allowed to dry at room temperature for 24 hours.

TABLE 1

| | Advancing/Receding | Advancing/Receding |
|---|---|---|
| | Contact Angle with | Contact Angle with |
| Example | water | hexadecane |
| Example 1 | 116/88 | 83/47 |
| Example 2 | 111/86 | 66/42 |
| Example 3 | 114/96 | 79/64 |
| Example 4 | 112/97 | 78/62 |
| Example 5 | 114/92 | 74/58 |
| Example 6 | 107/83 | 72/60 |
| Example 7 | 67/44 | 52/39 |
| Example 8 | 112/93 | 75/60 |
| Example 9 | 65/39 | 50/34 |
| Example 10 | 110/92 | 80/62 |
| Example 11 | 117/96 | 60/45 |
| Example 12 | 58/39 | 50/31 |
| Example 13 | 115/91 | 77/64 |
| Comp. Ex. A | 104/82 | 70/48 |
| Comp. Ex. B | 113/95 | 71/61 |

### Surface Tension Measurement

Examples 7 and 12 were diluted with deionized water to the concentrations given in Table 1 (below). Surface tensions were measured for the resulting solutions using a Kruss K-12 tensiometer (obtained from Kruss GmbH, Hamburg, Germany) using the Du Nouy ring method at 20° C. The results are summarized in Table 2 (below).

TABLE 2

| Example | Concentration (ppm) | Surface tension (mN/m) |
|---|---|---|
| 7 | 1000 | 24.7 |
| | 100 | 35.6 |
| | 10 | 55.9 |
| 12 | 1000 | 21.5 |
| | 100 | 32.7 |
| | 10 | 52.0 |

### Example 14

### Part A

CF₃CFH—O—(CF₂)₅COOH (426 grams, 1.0 mole), which was prepared according to the method described in Example 3 of U.S. Pat. App. Pub. No. 2007/0276103, was esterified at 65° C. with methanol (200 grams, 6.3 moles) and concentrated sulfuric acid (200 grams, 2.0 moles). The reaction mixture was washed with water and distilled at 172° C. to give 383 grams of CF₃CFH—O—(CF₂)₅COOCH₃, which was combined with material from a repeat run and used in Part B.

### Part B

A 5-L round-bottom flask equipped with a mechanical stirrer and nitrogen bubbler was charged with 1 L of 1,2-dimethoxyethane and sodium borohydride (76 grams, 2.0 moles) and heated to 80° C. CF₃CFH—O—(CF₂)₅COOCH₃ (713 grams, 1.67 mole), prepared as described in Part A, was added to the stirred slurry over a period of one hour. A mixture of concentrated sulfuric acid (198 grams) and water (1.0 L) was added to the reaction mixture. The lower phase was separated, and the solvent was removed by distillation. Further distillation provided 506 grams of CF₃CFH—O—(CF₂)₅CH₂OH (boiling point 173° C.), the structure of which was confirmed by Fourier Transform Infrared Spectroscopy (FTIR) and ¹H and ¹⁹F Nuclear Magnetic Resonance (NMR) Spectroscopy.

### Part C

A 100-mL flask equipped with a magnetic stirrer, thermometer, and condenser was charged with CF₃CFH—O—(CF₂)₅CH₂OH (7.3 grams, 18.4 mmol), a polyfunctional isocyanate compound made from hexamethylene-1,6-diisocyanate (obtained from Bayer Material Science under the trade designation “DESMODUR N-3300”) (3.5 grams, 18.3 milliequivalents of isocyanate), and ethyl acetate (25 grams) under a nitrogen atmosphere, and the mixture was stirred to give a homogeneous solution of about 30% by weight. Two drops of dibutyltin dilaurate were added, and the reaction mixture was heated at 60° C. for four hours under nitrogen using a heating mantle. Analysis by IR spectroscopy indicated no residual isocyanate was left.

### Example 15

Example 15 was prepared according to the method of Example 14, Part C except using 5.75 grams (14.4 mmol) of CF₃CFH—O—(CF₂)₅CH₂OH, 1.3 gram (4.8 mmol) of 1-octadecanol, 3.7 grams (19.2 isocyanate milliequivalents) of the polyfunctional isocyanate compound “DESMODUR N-3300”, and 25 grams of ethyl acetate.

### Example 16

Example 16 was prepared according to the method of Example 14, Part C except using 4.1 grams (10.3 mmol) of CF₃CFH—O—(CF₂)₅CH₂OH, 3.7 grams (10.3 mmol) of N-methylperfluorobutanesulfonamido ethanol, 3.9 grams (20.6 isocyanate milliequivalents) of the polyfunctional isocyanate compound “DESMODUR N-3300”, and 27 grams of ethyl acetate.

N-Methylperfluorobutanesulfonamido ethanol was prepared according to the method of Example 1 of U.S. Pat. No. 2,803,656 (Ahlbrecht et al.), the disclosure of which example is incorporated herein by reference.

### Example 17

Example 17 was prepared according to the method of Example 14, Part C except using 8.75 grams (22.0 mmol) of CF₃CFH—O—(CF₂)₅CH₂OH, 4.2 grams (22.0 isocyanate milliequivalents) of the biuret of hexamethylene-1,6-diisocyanate obtained from Bayer Material Science under the trade designation “DESMODUR N-100” instead of the polyfunctional isocyanate compound “DESMODUR N-3300”, and 30 grams of ethyl acetate.

### Example 18

### Part A

A 5-L round-bottom flask equipped with a mechanical stirrer and nitrogen bubbler was charged with a portion of the material from Example 14, Part B (250 grams, 0.63 mole), diisopropylethylamine (90 grams, 0.7 mole), and 200 grams tert-butyl methyl ether and heated at 55° C. for 30 minutes. Acryloyl chloride (61 grams, 0.67 mole) was added over a period of 30 minutes. During the addition, a slight reflux was maintained, and a precipitate formed. Water (15 grams), magnesium sulfate (16 grams), potassium carbonate (16 grams), and silica gel (90 grams) were added, and the resulting mixture was stirred for 15 minutes, vacuum filtered, and concentrated at 50° C./0.1 mmHg (13 Pa) to provide 250 grams (0.55 mole) of CF₃CFH—O—(CF₂)₅CH₂OC(O)CH═CH₂, the structure of which was confirmed by FTIR and ¹H and ¹⁹F NMR Spectroscopy.

### Part B

Under a nitrogen atmosphere, 0.05 gram 2,2′-azobis(2-methylbutyronitrile) (obtained from E.I. DuPont de Nemours & Co., Wilmington, Del., under the trade designation “VAZO 67”) was added to a solution of 4.5 grams, 10 mmol of CF₃CFH—O—(CF₂)₅CH₂OC(O)CH═CH₂ and 0.81 grams (2.5 mmol) octadecyl acrylate and 0.195 grams (2.5 mmol) of 2-mercaptoethanol in 13 grams of ethyl acetate. The reaction, which was approximately 30% by weight solids, was heated at 70 to 75° C. for 24 hours to form a hydroxyl-terminated oligomer. Analysis by IR spectroscopy indicated no residual acrylate groups remained. Then, 0.21 gram hexamethylene-1,6-diisocyanate (2.50 isocyanate milliequivalents) was added at room temperature under nitrogen, followed by the addition of two drops of dibutyltin dilaurate. The solution was heated at 70° C. for 4 hours, after which time, analysis by FTIR indicated no isocyanate groups remained.

### Dynamic Contact Angle Measurement for Examples 14 to 18

Nylon 66 film (obtained from E.I. DuPont de Nemours & Co.) was cut into strips, and the strips were cleaned with methyl alcohol. Using a smaller binder clip to hold one end of the nylon film, the strip was immersed in a treating solution (about 5% solids) and withdrawn slowly from the solution. The coated strip was allowed to air dry undisturbed for a minimum of 30 minutes and then was heated for 10 minutes at 150° C.

Advancing and receding contact angles on the coated film were measured using a CAHN Dynamic Contact Angle Analyzer, Model DCA 322 (a Wilhelmy balance apparatus equipped with a computer for control and data processing, obtained from ATI, Madison, Wis.). Water and hexadecane were used as probe liquids, and the average of 3 measurements are reported in Table 3, below.

TABLE 3

| Fluorochemical | Advancing/Receding CA | Advancing/Receding CA |
|---|---|---|
| material | with water | with hexadecane |
| Example 14 | 113/58 | 72/67 |
| Example 15 | 106/65 | 51/42 |
| Example 16 | 99/69 | 76/47.5 |
| Example 17 | 90/72 | 67/41 |
| Example 18 | 103/65 | 59/32 |

Various modifications and alterations of this disclosure may be made by those skilled in the art without departing from the scope and spirit of this disclosure, and it should be understood that this disclosure is not to be unduly limited to the illustrative embodiments set forth herein.

1. A compound comprising:
an end group represented by formula:
(Rf-Q)ₐ-X-(A-)_{b}; and
a multivalent unit comprising a segment represented by formula: 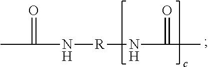
wherein
each Rf is independently:
Rf^{A}—(O)ᵣ—CHL′-(CF₂)ₙ—;
[Rf^{B}—(O)ₜ—C(L)H—CF₂—O]ₘ—W—;
CF₃CFH—O—(CF₂)ₚ—;
CF₃—(O—CF₂)_{z}—; or
CF₃—O—(CF₂)₃—O—CF₂—;
each Q is independently alkylene or arylalkylene, wherein alkylene and arylalkylene are optionally interrupted or terminated by at least one functional group that is independently ether, amine, ester, amide, carbamate, or urea, wherein when “a” is 1, Q may also be a bond, —C(O)O—, or —C(O)—N(R″)—;
X is alkylene or an alkylenic polymer backbone, each of which is optionally interrupted by —S— or —O—, wherein the alkylenic polymer backbone is optionally substituted with at least one alkyl ester group that is optionally substituted with —Si(G)₃, an ammonium group, a polyalkyleneoxy segment, a carboxylate, a sulfonate, a sulfate, a phosphate, or a phosphonate, wherein each G is independently hydroxyl, alkoxy, acyloxy, aryloxy, halogen, alkyl, or phenyl, and wherein at least one G is alkoxy, acyloxy, aryloxy, or halogen;
each A is independently —O—, —N(R¹)—, —S—, or —C(O)O—;
R″ and R¹ are each independently hydrogen or alkyl having up to 4 carbon atoms;
a is a number from 1 to 10;
b is a number from 1 to 5;
c is a number from 1 to 20;
Rf^{A} and Rf^{B} independently represent a partially or fully fluorinated alkyl group having from 1 to 6 carbon atoms and optionally interrupted with at least one oxygen atom;
L is F or CF₃;
L′ is F or H;
W is alkylene or arylene;
r is 0 or 1, wherein when r is 0, then Rf^{A} is interrupted with at least one oxygen atom;
t is 0 or 1;
m is 1, 2, or 3;
n is 0 or 1;
each p is independently a number from 1 to 6;
z is a number from 2 to 7; and
R is alkylene, arylene, or arylalkylene, each of which is optionally interrupted by at least one biruet, allophanate, uretdione, or isocyanurate linkage.

2. The compound according to claim 1, wherein the compound further comprises an end group represented by formula:
alkyl-A-;
Rf³-Q¹-A-;
alkyl-O-[EO]_{f}-[R²O]_{g}-[EO]_{f}—;
alkyl-O—[R²O]_{g}-[EO]_{f}—[R²O]_{g}—; 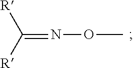
[(G)₃Si]_{d}—X′-A-;
(D)₁₋₃-R³-A-; or
(M)₁₋₂-R⁴-A-;
wherein
Rf³ is perfluoroalkyl having up to 6 carbon atoms and optionally interrupted by one or two —O— groups;
Q¹ is alkylene or arylalkylene, wherein alkylene and arylalkylene are optionally interrupted or terminated by at least one functional group that is independently ether, amine, ester, amide, sulfonamide, carbamate, or urea;
EO represents —CH₂CH₂O—;
each R²O independently represents —CH(CH₃)CH₂O—, —CH₂CH(CH₃)O—, —CH₂CH₂CH₂O—, —CH(CH₂CH₃)CH₂O—, —CH₂CH(CH₂CH₃)O—, —CH₂CH₂CH₂CH₂O—, or —CH₂C(CH₃)₂O—;
each f is independently a number from 1 to 150;
each g is independently a number from 0 to 55;
each G is independently hydroxyl, alkoxy, acyloxy, aryloxy, halogen, alkyl, or phenyl, wherein at least one G is alkoxy, acyloxy, aryloxy, or halogen;
each D is independently acrylate, methacrylate, epoxide, glycidoxy, or vinyl;
M is an ammonium group, a carboxylate, a sulfonate, a sulfate, a phosphate, or a phosphonate;
each R′ is independently hydrogen, alkyl, or aryl;
R³ is divalent, trivalent, or tetravalent alkylene;
R⁴ is alkylene that is optionally interrupted by at least one ether linkage or amine linkage;
X′ is alkylene or an alkylenic polymer backbone, each of which is optionally interrupted by —S— or —O—, wherein the alkylenic polymer backbone is optionally substituted with at least one alkyl ester group that is optionally substituted with an ammonium group, a carboxylate, a sulfonate, a sulfate, a phosphate, or a phosphonate; and
d is a number from 1 to 5.

3. The compound according to claim 1, wherein the compound is represented by formula: 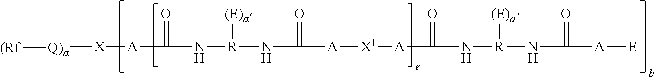
wherein
each a′ is independently 0, 1, or 2;
e is a number from 0 to 20;
X¹ is alkylene, polyalkyleneoxy, fluoroalkylene, or polyfluoroalkyleneoxy, wherein alkylene is optionally interrupted by at least one of —O—, polydialkylsiloxane, polydiarylsiloxane, or polyalkylarylsiloxane and is optionally substituted with —Si(G)₃, an ammonium group, a polyalkyleneoxy segment, a carboxylate, a sulfonate, a sulfate, a phosphate, or a phosphonate;
each E is independently an end group represented by formula:
(Rf-Q)ₐ-X-A-;
alkyl-A-;
Rf³-Q¹-A-;
alkyl-O-[EO]_{f}-[R²O]_{g}-[EO]_{f}—;
alkyl-O—[R²O]_{g}-[EO]_{f}—[R²O]_{g}—; 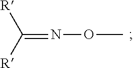
[(G)₃Si]_{d}—X′-A-;
(D)₁₋₃-R³-A-; or
(M)₁₋₂-R⁴-A-; wherein
Rf³ is perfluoroalkyl having up to 6 carbon atoms and optionally interrupted by one or two —O— groups;
Q¹ is alkylene or arylalkylene, wherein alkylene and arylalkylene are optionally interrupted or terminated by at least one functional group that is independently ether, amine, ester, amide, sulfonamide, carbamate, or urea;
EO represents —CH₂CH₂O—;
each R²O independently represents —CH(CH₃)CH₂O—, —CH₂CH(CH₃)O—, —CH₂CH₂CH₂O—, —CH(CH₂CH₃)CH₂O—, —CH₂CH(CH₂CH₃)O—, —CH₂CH₂CH₂CH₂O—, or —CH₂C(CH₃)₂O—;
each f is independently a number from 1 to 150;
each g is independently a number from 0 to 55;
each G is independently hydroxyl, alkoxy, acyloxy, aryloxy, halogen, alkyl, or phenyl, wherein at least one G is alkoxy, acyloxy, aryloxy, or halogen;
each D is independently acrylate, methacrylate, epoxide, glycidoxy, or vinyl;
M is an ammonium group, a carboxylate, a sulfonate, a sulfate, a phosphate, or a phosphonate;
each R′ is independently hydrogen, alkyl, or aryl;
R³ is divalent, trivalent, or tetravalent alkylene;
R⁴ is alkylene that is optionally interrupted by at least one ether linkage or amine linkage;
X′ is alkylene or an alkylenic polymer backbone, each of which is optionally interrupted by —S— or —O—, wherein the alkylenic polymer backbone is optionally substituted with at least one alkyl ester group that is optionally substituted with an ammonium group, a carboxylate, a sulfonate, a sulfate, a phosphate, or a phosphonate; and
d is a number from 1 to 5.

4. The compound according to claim 1, wherein the compound comprises segments represented by formula: 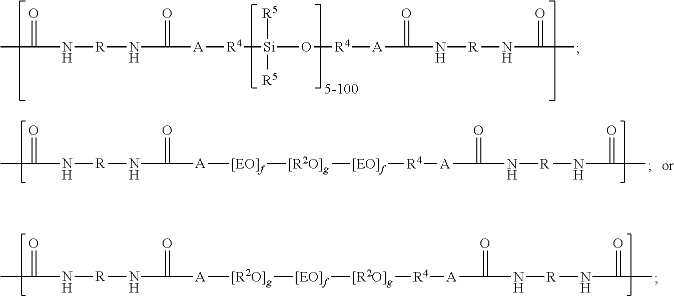
wherein
EO represents —CH₂CH₂O—;
each R²O independently represents —CH(CH₃)CH₂O—, —CH₂CH(CH₃)O—, —CH₂CH₂CH₂O—, —CH(CH₂CH₃)CH₂O—, —CH₂CH(CH₂CH₃)O—, —CH₂CH₂CH₂CH₂O—, or —CH₂C(CH₃)₂O—;
each f is independently a number from 1 to 150;
each g is independently a number from 0 to 55;
R⁴ is alkylene that is optionally interrupted by at least one ether linkage or amine linkage; and
each R⁵ is independently alkyl or aryl.

5. A compound comprising a reaction product of components comprising a multifunctional isocyanate compound and a fluorinated compound represented by formula:
(Rf-Q)ₐ-X—(Z)_{b}
wherein
each Rf is independently:
Rf^{A}—(O)ᵣ—CHL′—(CF₂)ₙ—;
[Rf^{B}—(O)ₜ—C(L)H—CF₂—O]ₘ—W—;
CF₃CFH—O—(CF₂)ₚ—;
CF₃—(O—CF₂)_{z}—; or
CF₃O—(CF₂)₃—O—CF₂—;
each Q is independently alkylene or arylalkylene, wherein alkylene and arylalkylene are optionally interrupted or terminated by at least one functional group that is independently ether, amine, ester, amide, carbamate, or urea, wherein when “a” is 1, Q may also be a bond, —C(O)O—, or —C(O)—N(R″)—, and wherein R″ is hydrogen or alkyl having up to 4 carbon atoms;
X is alkylene or an alkylenic polymer backbone, each of which is optionally interrupted by —S— or —O—, wherein the alkylenic polymer backbone is optionally substituted with at least one alkyl ester group that is optionally substituted with —Si(G)₃, an ammonium group, a polyalkyleneoxy segment, a carboxylate, a sulfonate, a sulfate, a phosphate, or a phosphonate, wherein each G is independently hydroxyl, alkoxy, acyloxy, aryloxy, halogen, alkyl, or phenyl, and wherein at least one G is alkoxy, acyloxy, aryloxy, or halogen;
each Z is independently hydroxyl, amino, mercaptan, isocyanate, epoxy, or a carboxylic acid;
a is a number from 1 to 10;
b is a number from 1 to 5;
Rf^{A} and Rf^{B} independently represent a partially or fully fluorinated alkyl group having from 1 to 6 carbon atoms and optionally interrupted with at least one oxygen atom;
L is F or CF₃;
L′ is F or H;
W alkylene or arylene;
r is 0 or 1, wherein when r is 0, then Rf^{A} is interrupted with at least one oxygen atom;
t is 0 or 1;
m is 1, 2, or 3;
n is 0 or 1;
each p is independently a number from 1 to 6; and
z is a number from 2 to 7.

6. The compound according to claim 5, wherein the components further comprise at least one of a fluorinated alcohol, fluorinated polyol, a non-fluorinated polyol, an aliphatic alcohol, a polyamine, a silane compound represented by formula [(G)₃Si]_{d}—X′—Z, an oxime, a polymerizable compound represented by formula (D)₁₋₃-R³—Z, or a compound represented by formula (M)₁₋₂-R⁴—Z, wherein
each G is independently hydroxyl, alkoxy, acyloxy, aryloxy, halogen, alkyl, or phenyl, wherein at least one G is alkoxy, acyloxy, aryloxy, or halogen;
X′ is alkylene or an alkylenic polymer backbone, each of which is optionally interrupted by —S— or —O—, wherein the alkylenic polymer backbone is optionally substituted with at least one alkyl ester group that is optionally substituted with an ammonium group, a carboxylate, a sulfonate, a sulfate, a phosphate, or a phosphonate;
each D is independently acrylate, methacrylate, epoxide, glycidoxy, or vinyl;
each M is independently an ammonium group, a carboxylate, a sulfonate, a sulfate, a phosphate, or a phosphonate;
R³ is divalent, trivalent, or tetravalent alkylene; and
R⁴ is alkylene that is optionally interrupted by at least one ether linkage.

7. The compound according to claim 5, wherein each Rf is independently:
Rf^{A}—(O)ᵣ—CHF—(CF₂)ₙ—;
[Rf^{B}—(O)ₜ—C(L)H—CF₂—O]ₘ—W—; or
CF₃CFH—O—(CF₂)ₚ—.

8. The compound according to claim 5, wherein t and r are each 1, and wherein Rf^{A} and Rf^{B} are independently selected from the group consisting of:
fully fluorinated alkyl groups having from 1 to 3 carbon atoms; and
fully fluorinated groups represented by formula:
R_{f}¹—[OR_{f}²]ₓ—,
wherein
R_{f}¹ is a perfluorinated alkyl group having from 1 to 3 carbon atoms;
each R_{f}² is independently perfluorinated alkylene having from 1 to 3 carbon atoms; and
x is an integer having a value from 1 to 4.

9. The compound according to claim 5, wherein t and r are each 0, and wherein Rf^{A} and Rf^{B} are independently a fully fluorinated group represented by formula:
R_{f}⁴—[OR_{f}⁵]_{y}—O—CF₂—,
wherein
R_{f}⁴ is a perfluorinated alkyl group having from 1 to 3 carbon atoms;
each R_{f}⁵ is independently perfluorinated alkylene having from 1 to 3 carbon atoms; and
y is an integer having a value from 0 to 4.

10. The fluorinated compound according to claim 5, wherein Rf is CF₃—O—CF₂CF₂—CF₂—O—CHF—; CF₃—O—CF₂—CF₂—CF₂—O—CF₂—CHF—; CF₃—O—CF₂—CF₂—CF₂—O—CHF—CF₂—; or CF₃—O—CF₂—CF₂—CF₂—O—CF₂—CHF—CF₂—.

11. The compound according to claim 5, wherein X is an alkylenic polymer backbone.

12. The compound according to claim 11, wherein (Rf-Q)ₐ-X— is represented by formula: 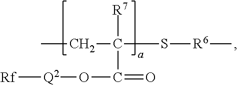
wherein
each Q² is independently —C(O)—N(R″)-alkylene, alkylene, —C(O)—N(R″)-arylalkylene, or arylalkylene, each of which is optionally interrupted by at least one ether linkage and wherein R″ is hydrogen or alkyl having up to 4 carbon atoms;
R⁶ is alkylene, arylene, or arylalkylene, each of which is divalent or trivalent and optionally interrupted by at least one ether linkage; and
each R⁷ is independently hydrogen or methyl.

13. A method of making an article having a surface, the method comprising treating the surface with a compound according to claim 5.

14. An article comprising a surface, wherein at least a portion of the surface is treated with a compound according to claim 5.

15. A method of reducing the surface tension of a liquid, the method comprising combining the liquid with an amount of a compound according to claim 5, wherein the amount of the compound is sufficient to reduce the surface tension of the liquid.

16. The compound according to claim 1, wherein each Rf is independently:
Rf^{A}—(O)ᵣ—CHF—(CF₂)ₙ—;
[Rf^{B}—(O)ₜ—C(L)H—CF₂—O]ₘ—W—; or
CF₃CFH—O—(CF₂)ₚ—.

17. The compound according to claim 1, wherein t and r are each 1, and wherein Rf^{A} and Rf^{B} are independently selected from the group consisting of:
fully fluorinated alkyl groups having from 1 to 3 carbon atoms; and
fully fluorinated groups represented by formula:
R_{f}¹—[OR_{f}²]ₓ—,
wherein
R_{f}¹ is a perfluorinated alkyl group having from 1 to 3 carbon atoms;
each R_{f}² is independently perfluorinated alkylene having from 1 to 3 carbon atoms; and
x is an integer having a value from 1 to 4.

18. The compound according to claim 1, wherein t and r are each 0, and wherein Rf^{A} and Rf^{B} are independently a fully fluorinated group represented by formula:
R_{f}⁴—[OR_{f}⁵]_{y}—O—CF₂—,
wherein
R_{f}⁴ is a perfluorinated alkyl group having from 1 to 3 carbon atoms;
each R_{f}⁵ is independently perfluorinated alkylene having from 1 to 3 carbon atoms; and
y is an integer having a value from 0 to 4.

19. The fluorinated compound according to claim 1, wherein Rf is CF₃—O—CF₂CF₂—CF₂—O—CHF—; CF₃—O—CF₂—CF₂—CF₂—O—CF₂—CHF—; CF₃—O—CF₂—CF₂—CF₂—O—CHF—CF₂—; or CF₃—O—CF₂—CF₂—CF₂—O—CF₂—CHF—CF₂—.

20. The compound according to claim 1, wherein X is an alkylenic polymer backbone.
